(19) United States
(12) Patent Application Publication
(10) Pub. No.: US 2025/0170212 A1
(43) Pub. Date: May 29, 2025

(54) COMPOSITIONS AND METHODS OF TREATING AMYOTROPHIC LATERAL SCLEROSIS (ALS) AND RELATED DISORDERS

(71) Applicant: BIOINCEPT, LLC, Cherry Hill, NJ (US)

(72) Inventor: Eytan R. Barnea, Cherry Hill, NJ (US)

(21) Appl. No.: 18/834,179

(22) PCT Filed: Feb. 28, 2023
(86) PCT No.: PCT/US2023/014195

§ 371 Date: Jul. 29, 2024

Related U.S. Application Data
(60) Provisional application No. 63/268,670, filed on Feb. 28, 2022.

(51) Int. Cl. A61K 38/17; A61K 31/4152; A61K 31/428; A61P 25/28

(52) U.S. Cl. CPC A61K 38/1709; A61K 31/4152; A61K 31/428; A61P 25/28

(57) ABSTRACT

The present invention relates to methods for the treatment or prevention of symptoms associated with amyotrophic lateral sclerosis (ALS) and related disorders. The present disclosure provides compositions and methods for preventing or treating traumatic brain injuries and other neurological disorders arising from such an injury in a subject by administering to the subject an effective amount of a composition comprising PIF. In certain embodiments, the present disclosure provides compositions and methods for treating traumatic brain injuries and other neurological disorders arising from such an injury in a subject by administering to the subject an effective amount of a composition comprising PIF co-administered with additional agents or treatments.

### REFERENCE TO RELATED APPLICATIONS

This application is a U.S. National Stage filing under 35 USC 371 of International Application No. PCT/US2023/014195, filed on Feb. 28, 2023, which claims the benefit of U.S. Provisional Patent Application No. 63/268,670, filed Feb. 28, 2022, which is incorporated by reference herein in its entirety.

### REFERENCE TO A SEQUENCE LISTING

This application contains a Sequence Listing submitted electronically as a text file by EFS-Web. The text file, named “45884-0135_Sequence_Listing_20230228.txt” has a byte size of 12 KB, and was recorded on 2023 Feb. 27. The information contained in the text file is incorporated herein by reference in its entirety pursuant to 37 CFR 1.52(e)(5).

### BACKGROUND OF THE INVENTION

### Field of the Invention

The present invention, in at least some aspects, relates to compositions and methods of treatment for motor neuron diseases (MND) and in particular to compositions containing an inventive molecule as described herein and methods of treatment using same. The present invention also relates to a pharmaceutical agent for treating amyotrophic lateral sclerosis (hereinafter also referred to as “ALS”) or suppressing the disease progress thereof, or treating symptoms caused by ALS or suppressing the progress of symptoms thereof. The invention provides a method for treating ALS or suppressing the disease progress thereof, or treating symptoms caused by ALS or suppressing the disease progress thereof by administering an agent containing, as an active ingredient, pre-implantation factor (PIF) mutants, to a patient.

### Description of the Related Art

Amyotrophic lateral sclerosis (ALS), which is one type of motor neuron disease, is an intractable disease characterized by degeneration of the upper and lower motor neurons of the cortex, the brain stem, and the spinal cord. Although the start is focal, the heterogeneous manifestations and disseminating patterns suggest the spreading among advancement motor neuronal pools with glial partners. The end stage of ALS is the respiratory failure (about 3-4 years after the onset) and currently no effective cure is available to stop or delay the disease from progression. Therefore, testing of new therapeutic approaches is essential and one well-defined animal model is the SOD1^{G93A} transgenic mouse model. In this model, mice express a G93A mutant form of human SOD1 and develop progressive loss of upper and lower motor neurons. Typically, within 4-5 month of symptom onset mice express muscle denervation, weakness, and atrophy resulting in death.

The importance of glial cells in the degeneration of motor neurons emerged after synthesis of SOD1 mutants and consecutive selective silencing. The activation of innate immune cells such as microglia and excessive extracellular production of superoxide contribute substantially to ALS development and progression. Not surprisingly, the transplantation of murine glial-restricted precursors (mGRPs) is an attractive strategy to modulate ALS development. Notably, GRPs are oligodendrocytic and astrocytic cell lineage precursors and recent advancements support its clinical treatments for ALS. However, the optimal therapeutic window, the ideal cell type and dose, and delivery site or method are still unclear. The recently updated Cochrane review underlines the need of further research to explore combination of cellular therapy and novel therapeutics. Such adjuvant therapeutics may include synthetic PreImplantation Factor (SPIF), Takrolimus (Tac), and Costimulatory Blockade (CB).

PIF is a small 15-amino acid pregnancy derived peptide, which is secreted by the trophoblast/embryo. Besides immunomodulatory properties in and outside pregnancy, a synthetic version of PIF (PIF analog: sPIF) was successfully tested in animal models of multiple immune disorders and received a Fast-Track FDA approval (autoimmune diseases of non-pregnant subjects-clinicaltrials.gov, NCT02239562). sPIF was able to reverse and prevent paralysis and restore myelination through inhibiting neuro-inflammation in murine models of experimental autoimmune encephalomyelitis. The neuroprotective property of sPIF was further underscored by its ability to mitigate neuronal loss and microglial activation in murine model of immature brain injury as well. Tac is a calcineurin inhibitor and a first-line immunosuppressive drug used after organ transplant, which additionally may enhance neuronal regeneration. Finally, the T cells feature crucial immune response toward allografts and co-stimulatory molecules involved in T-cell activation may be targeted as well. The so called costimulatory blockade (CB) includes CTLA4 (Cytotoxic T-lymphocyte-associated antigen 4) and MR-1 (anti-CD154 antibody/CD40L monoclonal antibody) targeting CD40/CD154 axis.

At present, as a pharmaceutical product effective for suppression of the progress of ALS, there has been approved riluzole, which suppresses glutamic acid transport in the glutamatergic nerve. However, it has been reported that the effectiveness of riluzole could not be confirmed. There is therefore a need in the art for the discovery of additional methods for the treatment or prevention of symptoms associated with amyotrophic lateral sclerosis (ALS) and related disorders. The present disclosure describes compositions and methods of using PIF alone and in combinations to treat amyotrophic lateral sclerosis (ALS) and related disorders.

### SUMMARY OF THE INVENTION

In one or more embodiments, the present invention provides compositions comprising inventive molecules as described herein and methods of treatment with same. By “inventive molecule” it is meant a preimplantation factor (PIF) peptide, or active analog thereof which, as described herein, has been shown to have at least one effect in vitro and/or in vivo, that indicates that it would be useful in the compositions and methods of treatment described herein.

In one or more embodiments, the treatment comprises one or more of curing, managing, reversing, attenuating, alleviating, minimizing, suppressing, managing, or halting the deleterious effects of the above-described diseases.

According to at least some embodiments, treating also includes at least reducing the rate of onset of symptoms and/or etiology of the disease, for example optionally as determined by measurement of one or more diagnostic markers.

With regard to the inventive molecules as described herein, without wishing to be limited by a single hypothesis, it is possible that for each disease described herein, prevention or delay of full onset or even symptomatic presentation of these diseases in subjects without symptoms of the disease, or with only minor initial symptoms would be possible by detecting the disease in the subject before full onset or symptomatic presentation, and then administering the inventive molecules as described herein to the subject according to a suitable dosing regimen.

In one embodiment, managing comprises reducing the severity of the disease, reducing the frequency of episodes of the disease, reducing the duration of such episodes, or reducing the severity of such episodes or a combination thereof.

Individuals at risk of developing a disease can be identified based on various approaches either before disease development or at very early stages in which disease markers can be identified. The identification of individuals at risk as well as diagnosis of early disease can rely on various approaches including genomics, proteomics, metabolomics, lipidomics, glycomics, secretomics, serologic approaches and also optionally tests involving impairment of information processing. Family history can also provide information either in combination with one of the previously described approaches or as a standalone approach. Furthermore, over the past decade microbiome composition is becoming recognized as an important factor in health and disease.

Compositions and methods for the treatment or prevention of symptoms associated with amyotrophic lateral sclerosis (ALS) and related disorders, are provided. The present invention relates to compositions and methods for treating amyotrophic lateral sclerosis (ALS) and related disorders. The methods of the present invention are intended for the alleviation of symptoms associated with amyotrophic lateral sclerosis (ALS) and related disorders.

The present disclosure relates to a method of treating or preventing traumatic injury of the central nervous system in a subject in need thereof, the method comprising administering to the subject at least one pre-implantation factor (PIF) peptide, an analog thereof, or a pharmaceutically acceptable salt thereof.

In some embodiments, the step of administering to the subject at least one PIF peptide, an analog thereof, or a pharmaceutically acceptable salt thereof comprises administering a therapeutically effective dose of the at least one PIF molecule, an analog thereof, or a pharmaceutically acceptable salt thereof.

In some embodiments, the step of administering to the subject at least one PIF peptide, an analog thereof, or a pharmaceutically acceptable salt thereof comprises administering a therapeutically effective dose of the PIF peptide, an analog thereof, or pharmaceutically acceptable salt thereof from about 0.001 mg/kg to about 200 mg/kg.

In some embodiments, the step of administering to the subject at least one PIF peptide, an analog thereof, or a pharmaceutically acceptable salt thereof comprises administering a therapeutically effective dose of the PIF peptide, an analog thereof, or pharmaceutically acceptable salt thereof from about 0.1 mg/kg to about 5 mg/kg.

In some embodiments, the step of administering to the subject at least one PIF peptide, an analog thereof, or a pharmaceutically acceptable salt thereof comprises administering a therapeutically effective dose of the PIF peptide, an analog thereof, or pharmaceutically acceptable salt thereof from about 0.1 mg/kg to about 2.5 mg/kg/day or more subcutaneous (SQ or Sub-Q).

In some embodiments, the step of administering to the subject at least one PIF peptide, an analog thereof, or a pharmaceutically acceptable salt thereof comprises administering a therapeutically effective dose of the PIF peptide, an analog thereof, or pharmaceutically acceptable salt thereof from about 0.1 mg/kg to about 2.5 mg/kg/day or more subcutaneous (SQ or Sub-Q) for daily for 1, 2, 3, 4, 5, 6 days or more.

In some embodiments, the step of administering to the subject at least one PIF peptide, an analog thereof, or a pharmaceutically acceptable salt thereof comprises administering a therapeutically effective dose of the PIF peptide, an analog thereof, or pharmaceutically acceptable salt thereof from about 0.1 mg/kg to about 2.5 mg/kg/day or more subcutaneous (SQ or Sub-Q) daily for 1, 2, 3, 4, 5, 6 weeks or more.

In some embodiments, the step of administering to the subject at least one PIF peptide, an analog thereof, or a pharmaceutically acceptable salt thereof comprises administering a therapeutically effective dose of the PIF peptide, an analog thereof, or pharmaceutically acceptable salt thereof from about 0.1 mg/kg to about 2.5 mg/kg/day or more subcutaneous (SQ or Sub-Q) daily for 1, 2, 3, 4, 5, 6 months or more.

In some embodiments, the method further comprises administering at least one analgesic and/or one anti-inflammatory compound. In some embodiments, the method further comprises administering at least one analgesic and or one anti-inflammatory compound before, after, or simultaneously with the administration of a therapeutically effective dose of at least one PIF peptide, an analog thereof or pharmaceutically acceptable salt thereof.

In some embodiments, the therapeutically effective dose is from about 1.0 mg/kg to about 12.5 mg/kg/day, wherein kg is kilograms of the subject and mg is milligrams of the therapeutically effective dose. In some embodiments, the therapeutically effective dose is from about 1.0 mg/kg/day to about 1.5 mg/kg, wherein kg is kilograms of the subject and mg is milligrams of the therapeutically effective dose.

In some embodiments, the PIF peptide comprises one or more peptides selected from the group consisting of SEQ ID NO:1, SEQ ID NO:2, SEQ ID NO:3. In some embodiments, the PIF peptide comprises one or more peptides selected from the group consisting of SEQ ID NOs: 1-40 or a pharmaceutically acceptable salt thereof.

In some embodiments, the PIF peptide is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or analogs thereof, and combinations thereof. In certain embodiments, the PIF peptide is selected from the group consisting of SEQ ID NO: 1; SEQ ID NO: 2; SEQ ID NO: 3; SEQ ID NO: 4, and combinations thereof. In the some embodiments, the PIF peptide may be selected from compounds having amino acid structural and functional analogs, for example, peptidomimetics having synthetic or non-natural amino acids or amino acid analogues, so long as the mimetic has one or more functions or activities of compounds of the disclosure.

The present disclosure also relates to a method of treating or preventing amyotrophic lateral sclerosis in a subject in need thereof, the method comprising administering to the subject at least one pharmaceutical composition comprising: pre-implantation factor (PIF) peptide, an analog thereof, or a pharmaceutically acceptable salt thereof; and a pharmaceutically acceptable carrier. In some embodiments, the pharmaceutically acceptable carrier is sterile and pyrogen-free water.

In some embodiments, the therapeutically effective dose is about 0.5 mg/kg, wherein kg is kilograms of the subject and mg is milligrams of the therapeutically effective dose. In some embodiments, the therapeutically effective dose is about 1.0 mg/kg, wherein kg is kilograms of the subject and mg is milligrams of the therapeutically effective dose. In some embodiments, the therapeutically effective dose is about 1.5 mg/kg, wherein kg is kilograms of the subject and mg is milligrams of the therapeutically effective dose. In some embodiments, the therapeutically effective dose is about 2.0 mg/kg, wherein kg is kilograms of the subject and mg is milligrams of the therapeutically effective dose. In some embodiments, the therapeutically effective dose is about 2.5 mg/kg, wherein kg is kilograms of the subject and mg is milligrams of the therapeutically effective dose. In some embodiments, the therapeutically effective dose is about 3.0 mg/kg, wherein kg is kilograms of the subject and mg is milligrams of the therapeutically effective dose. In some embodiments, the therapeutically effective dose is about 4.0 mg/kg, wherein kg is kilograms of the subject and mg is milligrams of the therapeutically effective dose. In some embodiments, the therapeutically effective dose is about 0.2 mg/kg, wherein kg is kilograms of the subject and mg is milligrams of the therapeutically effective dose. In some embodiments, the therapeutically effective dose is about 0.3 mg/kg, wherein kg is kilograms of the subject and mg is milligrams of the therapeutically effective dose. In some embodiments, the therapeutically effective dose is about 0.4 mg/kg, wherein kg is kilograms of the subject and mg is milligrams of the therapeutically effective dose. In some embodiments, the therapeutically effective dose is about 0.5 mg/kg, wherein kg is kilograms of the subject and mg is milligrams of the therapeutically effective dose. In some embodiments, the therapeutically effective dose is about 0.6 mg/kg, wherein kg is kilograms of the subject and mg is milligrams of the therapeutically effective dose. In some embodiments, the therapeutically effective dose is about 0.7 mg/kg, wherein kg is kilograms of the subject and mg is milligrams of the therapeutically effective dose. In some embodiments, the therapeutically effective dose is about 0.8 mg/kg, wherein kg is kilograms of the subject and mg is milligrams of the therapeutically effective dose.

The present disclosure also relates to a pharmaceutical composition comprising (i) a therapeutically effective dose of one or a combination of PIF peptide or analogs thereof or pharmaceutically acceptable salts thereof; and (ii) a pharmaceutically acceptable carrier. In some embodiments, the pharmaceutically acceptable carrier is sterile and pyrogen-free water or Lactated Ringer's solution.

In some embodiments, the composition further comprises a therapeutically effective dose of one or a plurality of active agents. In some embodiments, the one or plurality of active agents is one or a combination of compounds chosen from: an anti-inflammatory compound, alpha-adrenergic agonist, antiarrhythmic compound, analgesic compound, and an anesthetic compound.

In some embodiments, the therapeutically effective dose of one or a combination of PIF peptide or analogs thereof or pharmaceutically acceptable salts thereof is about 1, 2, 3, 4, 5, 6, 7, 8, 9, 10 mg/kg or more, wherein kg is kilograms of the subject and mg is milligrams of the therapeutically effective dose.

In some embodiments, wherein the therapeutically effective dose of one or a combination of PIF peptide or analogs thereof or pharmaceutically acceptable salts thereof is about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2 mg/kg or more, wherein kg is kilograms of the subject and mg is milligrams of the therapeutically effective dose.

In some embodiments, wherein the therapeutically effective dose of one or a combination of PIF peptide or analogs thereof or pharmaceutically acceptable salts thereof is about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2 mg/kg/day or more subcutaneous (SQ or Sub-Q), wherein kg is kilograms of the subject and mg is milligrams of the therapeutically effective dose.

In some embodiments, the composition further comprises one or a plurality of stem cells. In some embodiments, the stem cell is an autologous stem cell.

In some embodiments, the pharmaceutical composition is administered via parenteral injection, subcutaneous injection, intravenous injection, intramuscular injection, intraperitoneal injection, transdermally, orally, buccally, ocular routes, intravaginally, by inhalation, by depot injections, or by implants.

In some embodiments, the compositions further comprise one or a combination of active agents chosen from: an anti-inflammatory compound, alpha-adrenergic agonist, antiarrhythmic compound, analgesic compound, and an anesthetic compound.

Within additional aspects of the invention, combinatorial formulations are provided which employ PIF and psychotherapeutic agents effective to prevent, treat, ameliorate, alleviate or reduce the amyotrophic lateral sclerosis (ALS) and related disorders in the subject, including human subjects. Exemplary combinatorial formulations and coordinate treatment methods employ coordinately administering (a) one or more additional therapeutic agent in an amount effective to prevent, ameliorate or alleviate one or more symptoms of the disorder, (b) coordinately administering one or more non-pharmacological treatments effective to prevent, ameliorate or alleviate one or more symptoms of the disorder, or (c) both. In one or more embodiments, the one or more additional therapeutic agent is selected from the group consisting of anti-depressant, mood-stabilizing, anxiolytic, anticonvulsant, stimulant, antipsychotic, antiaddictive, appetite suppressant drugs and opiate agonists.

In the coordinate administration methods of the invention, the PIF and the additional therapeutic agent are administered concurrently or sequentially in any order to prevent or treat one or more symptoms of the targeted disorders including amyotrophic lateral sclerosis (ALS) and related disorders. When administered simultaneously, the PIF and the psychotherapeutic agent may be combined in a single composition or combined dosage form, or administered at the same time in separate dosage forms.

The methods, formulations and coordinate treatment methods of the invention are effective to modulate, alleviate, treat or prevent one or more symptom(s) of the amyotrophic lateral sclerosis (ALS) and related disorders in a subject, including a mammalian subject. Such formulations and coordinate treatment methods may be administered prior to or shortly after a triggering event, or after development of symptoms of disorders including amyotrophic lateral sclerosis (ALS) and related disorders.

In order to determine whether an individual is at risk of acquiring amyotrophic lateral sclerosis (ALS) and related disorders and is therefore a candidate for preventative treatment with the present compositions and/or compounds, the individual's current life situation can be assessed.

If an individual exhibits the appropriate combination of symptoms indicating a diagnosis of amyotrophic lateral sclerosis (ALS) and related disorders as outlined above, then that individual can be treated with the present compounds and/or compositions.

Amyotrophic lateral sclerosis (ALS) and related disorders can be prevented or treated by administering therapeutically effective amounts of one or more of the present compounds and/or pharmaceutical compositions to a patient in need thereof. The present compounds and/or compositions are administered to a patient in a quantity sufficient to treat or prevent the symptoms and/or the underlying etiology associated with amyotrophic lateral sclerosis (ALS) or related disorders in the patient. The present compounds can also be administered in combination with other treatments and agents known to be useful in the treatment of amyotrophic lateral sclerosis (ALS) or related disorders, either in physical combination or in combined therapy through the administration of the present compounds and agents in succession (in any order).

Depending upon the particular needs of the individual subject involved, the present compounds can be administered in various doses to provide effective treatments for amyotrophic lateral sclerosis (ALS) or related disorders. Factors such as the activity of the selected compound, half-life of the compound, the physiological characteristics of the subject, the extent or nature of the subject's disease or condition, and the method of administration will determine what constitutes an effective amount of the selected compounds. Generally, initial doses will be modified to determine the optimum dosage for treatment of the particular subject. The compounds can be administered using a number of different routes including oral administration, topical administration, transdermal administration, intraperitoneal injection, or intravenous injection directly into the bloodstream. Effective amounts of the compounds can also be administered through injection into the cerebrospinal fluid or infusion directly into the brain, if desired.

These and other features are explained more fully in the embodiments illustrated below. It should be understood that in general the features of one embodiment also may be used in combination with features of another embodiment and that the embodiments are not intended to limit the scope of the invention.

### BRIEF DESCRIPTION OF THE FIGURES

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1. Experimental setup and clinical outcomes. A: Cartoon summarizing the experimental setup and treatment groups in the ALS mouse model (n=6 each group). The timing and duration of immunomodulatory drug applications are shown in green and GRPs transplantation in red. All animals received GRPs except Injury group, which received a sham transplantation. B: Kaplan-Meier curves indicating survival by group. sPIF transplantation seems superior but did not reach statistical significance. C: Functional assessments using hanging wire test by group. sPIF treatment resulted in significant improvement of motor outcome. mGRPs: glial-restricted precursors; sPIF: synthetic PreImplantation Factor; Tac: Tacrolimus, CB: co-stimulatory blockade. Differences between groups were analyzed using a repeated measure ANOVA model or using a log-rank test.

FIG. 2. Graft survival and peripheral immune responses. A: Representative bioluminescent images of the transplanted mGRPs and (B) graft survival over time. The immune modulators didn't impact the graft survival. C: Peripheral cytokines measured over time by groups with expected reduction of pro-inflammatory cytokine levels by additional use of immune modulators. mGRPs: glial-restricted precursors; sPIF: synthetic PreImplantation Factor; Tac: Tacrolimus, CB: co-stimulatory blockade. *p<0.05; **p<0.01; ***p<0.001. Data are presented as mean±SD (one-way analysis of variance (ANOVA) followed by a post-hoc test (Bonferroni test).

FIG. 3. Central immune responses. A: Representative images of microglial cells (Iba-1 positive cells) in two regions of the brain. B: All therapeutic groups reduced the number of microglial cells significantly. mGRPs: glial-restricted precursors; sPIF: synthetic PreImplantation Factor; Tac: Tacrolimus, CB: co-stimulatory blockade. *p<0.05; **p<0.01; ***p<0.001. Differences are calculated using one-way analysis of variance (ANOVA) followed by a post-hoc test (Bonferroni test).

FIG. 4. Neuronal survival. A: Representative images of mature neurons cells (NeuN positive cells) in two regions of the brain. B: All therapeutic groups reduced the number of microglial cells significantly. mGRPs: glial-restricted precursors; sPIF: synthetic PreImplantation Factor; Tac: Tacrolimus, CB: co-stimulatory blockade. *p<0.05; **p<0.01; ***p<0.001. Differences are calculated using one-way analysis of variance (ANOVA) followed by a post-hoc test (Bonferroni test).

FIG. 5. mGRPs progeny in brain parenchyma. Representative immunofluorescence images of astrocytes (GFAP+ red marker) and mGRPs (GFP+ green marker) in the diencephalon on day 56. Green arrows indicate positive cells in the sPIF group. mGRPs: glial-restricted precursors; SPIF: synthetic PreImplantation Factor; Tac: Tacrolimus, CB: co-stimulatory blockade.

FIG. 6. mGRPs progeny in brain parenchyma: Representative immunofluorescence images of oligodendrocytes (OLIG2+ red marker) and mGRPs (GFP+ green marker) in the diencephalon on day 56. Green arrows indicate positive cells in the sPIF group. mGRPs: glial-restricted precursors; sPIF: synthetic PreImplantation Factor; Tac: Tacrolimus, CB: co-stimulatory blockade.

FIG. 7. Immunofluorescence staining of mGRP cells (GFP+ green marker) and astrocytes (GFAP+ red marker) on whole cross section of the mouse diencephalon in the study groups. There can be seen a significantly larger area of astrocytes in the group of mice with sPIF administration (indicated by red arrows).

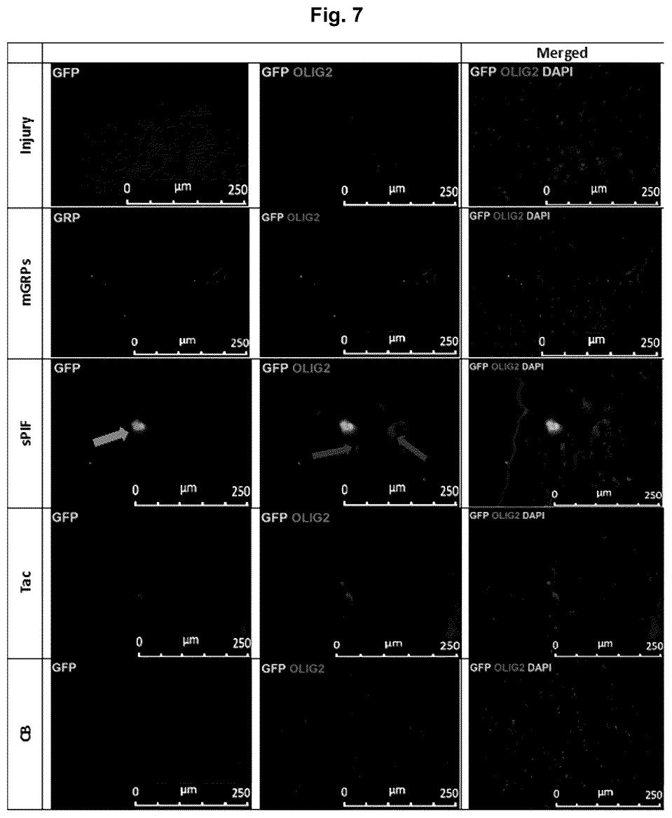

FIG. 8. Immunofluorescence staining of mGRP cells (GFP+ green marker) and oligodendrocytes (OLIG2+ red marker) on whole cross section of the mouse diencephalon in the study groups. There can be seen a larger area of oligodendrocytes in the group with sPIF administration (indicated by red arrows).

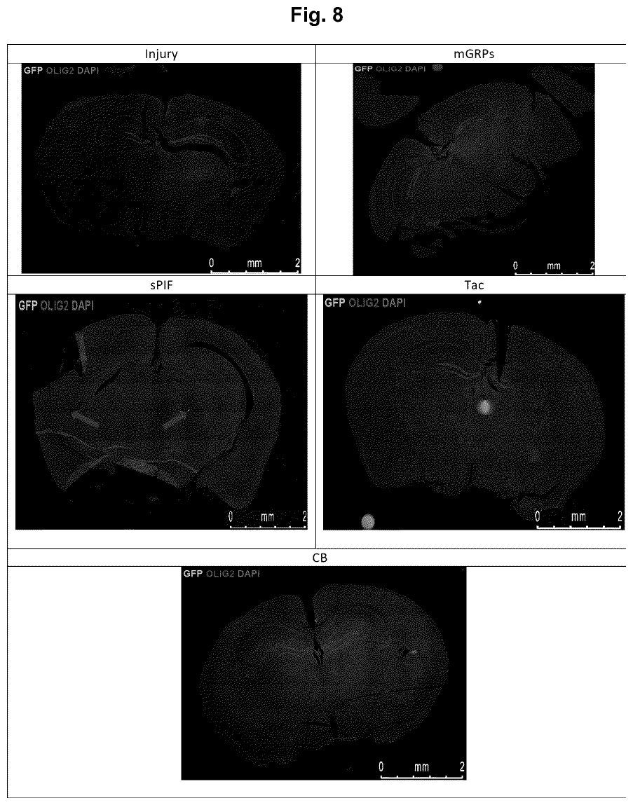

### DETAILED DESCRIPTION

### Definitions

As used herein, an “active therapeutic agent” of the present invention includes a preimplantation factor (PIF) peptide, or active analog thereof, and optionally includes one or more other therapeutic compound.

The term “administration” or “administering” includes routes of introducing the compounds, or a composition thereof, of the invention to a subject to perform their intended function. In one or more embodiments, administering includes when used in conjunction with a therapeutic means to administer a therapeutic directly into or onto a target tissue or to administer a therapeutic to a patient. Thus, as used herein, the term “administering”, when used in conjunction with PIF, can include, but is not limited to, providing PIF peptide into or onto the target tissue; providing PIF peptide systemically to a patient by, e.g., intravenous or subcutaneous injection; providing PIF peptide in the form of a nucleic acid molecule sequence that encodes PIF (e.g., by so-called gene-therapy techniques). “Administering” a composition may be accomplished by parenteral, oral or topical administration or any other suitable route.

The term “analog” refers to any peptidomimetic, functional fragment, mutant, variant, salt, pharmaceutical acceptable salt, polymorph, or non-naturally occurring peptide that is structurally similar to a naturally occurring full-length protein and shares at least one biochemical or biological activity of the naturally occurring full-length protein upon which the analog is based. In some embodiments, the term “analog” refers to any polypeptide comprising at least one a-amino acid and at least one non-native amino acid residue, wherein the polypeptide shares at least one biochemical or biological activity of the naturally occurring full-length protein upon which the analog is based. For instance in the case of PIF, a PIF analog may be 70% or more homologous to wild-type PIF and may share at least one binding property of wild-type PIF. PIF is known to bind to multiple receptors. Therefore, in some embodiments, the analog refers to a PIF peptidomimetic, functional fragment, mutant, variant, salt, polymorph, or non-naturally occurring peptide that is structurally similar to wild-type PIF but binds only to one of the naturally occurring ligands to which naturally occurring PIF binds.

The terms “carrier” or “vehicle” as used herein refer to carrier materials suitable for transdermal drug administration. Contemplated carriers and/or vehicles include any such materials known in the art, which are substantially nontoxic and/or do not interact with other components of a pharmaceutical formulation or drug delivery system in a deleterious manner.

The terms “composition” and “pharmaceutical composition” as used herein are equivalent terms referring to a composition of matter for pharmaceutical use.

The term “component” as used herein refers an ingredient that is combined with additional components/ingredients to obtain a composition.

“Disease” or “disorder” refers to an impairment of the normal function of an organism. As used herein, a disease may be characterized by the levels of primary or secondary injury causing the impairment of normal function.

The term “effective amount” or “therapeutically effective amount” as used herein means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment. For example, as used herein an “effective amount of the composition” is optionally the amount of composition that is sufficient to treat a subject who has suffered one or more motor neuron disease (MND) and in particular amyotrophic lateral sclerosis (ALS).

“Immune-modulating” refers to the ability of a compound of the present disclosure to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including lymphokines and antibodies, that have autocrine, paracrine, and endocrine effects on immune cells.

“Inflammatory response” or “inflammation” is a general term for the local accumulation of fluid, plasma proteins, and white blood cells initiated by physical injury, infection, or a local immune response. Inflammation is an aspect of many diseases and disorders, including but not limited to diseases related to immune disorders, viral infection, arthritis, autoimmune diseases, collagen diseases, allergy, asthma, pollinosis, and atopy. Inflammation is characterized by rubor (redness), dolor (pain), calor (heat) and tumor (swelling), reflecting changes in local blood vessels leading to increased local blood flow which causes heat and redness, migration of leukocytes into surrounding tissues (extravasation), and the exit of fluid and proteins from the blood and their local accumulation in the inflamed tissue, which results in swelling and pain, as well as the accumulation of plasma proteins that aid in host defense. These changes are initiated by cytokines produced by activated macrophages. Inflammation is often accompanied by loss of function due to replacement of parenchymal tissue with damaged tissue (e.g., in damaged myocardium), reflexive disuse due to pain, and mechanical constraints on function, e.g., when a joint swells during acute inflammation, or when scar tissue bridging an inflamed joint contracts as it matures into a chronic inflammatory lesion. In some embodiments, inflammation is caused or induced by pathogens, either directly or due to a local immune response. In the central nervous system, pathogens and pathogen induced inflammation is believed to be an underlying cause of many brain and spinal cord disorders. Pathogen induced inflammation may be caused by both bacterial and viral pathogens. “Anti-inflammatory” means regulation of inflammation not only anti-inflammatory refers to the ability of a compound to prevent or reduce the inflammatory response, or to soothe inflammation by reducing the symptoms of inflammation such as redness, pain, heat, or swelling. Inflammatory responses can be triggered by injury, for example injury to skin, muscle, tendons, or nerves. Inflammatory responses can also be triggered as part of an immune response. Inflammatory responses can also be triggered by infection, where pathogen recognition and tissue damage can initiate an inflammatory response at the site of infection. Generally, infectious agents induce inflammatory responses by activating innate immunity. Inflammation combats infection by delivering additional effector molecules and cells to augment the killing of invading microorganisms by the front-line macrophages, by providing a physical barrier preventing the spread of infection, and by promoting repair of injured tissue. “Inflammatory disorder” is sometimes used to refer to chronic inflammation due to any cause.

As used herein, the phrase “in need thereof” means that the animal or mammal has been identified or suspected as having a need for the particular method or treatment. In some embodiments, the identification can be by any means of diagnosis or observation. In any of the methods and treatments described herein, the animal or mammal can be in need thereof. In some embodiments, the animal or mammal is in an environment or will be traveling to an environment in which a particular disorder or condition is prevalent or more likely to occur.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

As used herein, the terms “peptide,” “polypeptide” and “protein” are used interchangeably and refer to two or more amino acids covalently linked by an amide bond or non-amide equivalent. The peptides of the disclosure can be of any length. For example, the peptides can have from about two to about 100 or more residues, such as, 5 to 12, 12 to 15, 15 to 18, 18 to 25, 25 to 50, 50 to 75, 75 to 100, or more in length. Preferably, peptides are from about 2 to about 18 residues. The peptides of the disclosure include 1- and d-isomers, and combinations of 1- and d-isomers. The peptides can include modifications typically associated with post-translational processing of proteins, for example, cyclization (e.g., disulfide or amide bond), phosphorylation, glycosylation, carboxylation, ubiquitination, myristylation, or lipidation.

The terms “pharmaceutically acceptable” or “pharmacologically acceptable” refer to molecular entities and compositions that do not produce an adverse, allergic or other untoward reaction when administered to an animal, or to a human, as appropriate. The term “pharmaceutically acceptable carrier” includes any and all solvents, dispersion media, coatings, antibacterial and antifungal agents, isotonic and absorption delaying agents and the like. The use of such media and agents with pharmaceutical active agents is well known in the art. In some embodiments, supplementary active ingredients can also be incorporated into the compositions.

A “polymorph” refers to solid crystalline forms of a compound. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Different physical properties of polymorphs can affect their processing.

In some embodiments, the terms “preimplantation factor” and “PIF” refer to PIF-1(15), a 15 amino acid peptide secreted by a human embryo prior to implantation. In some embodiments, PIF is secreted only by viable embryos. It is secreted by the fetus and the placenta, and can be detected in the maternal circulation; its presence in the maternal circulation significantly correlates with live birth. PIF plays an essential role in promoting implantation by acting on the decidua, modulating local immunity, enhancing embryo-decidual adhesion, and controlling apoptosis. Beyond promoting implantation and trophoblast invasion, PIF also has autotrophic protective effects on the embryo, promoting development and negating the toxicity of serum derived from patients with a history of recurrent pregnancy loss (RPL). In addition, PIF has shown an immunomodulatory effect in a juvenile mouse model of diabetes, wherein it modulates systemic Th1/Th2 cytokines and prevents diabetes development long-term. In an autoimmune encephalitis model, PIF reverses advanced paralysis, downregulates neural proinflammatory Th1-type genes and proteins, and inhibits IL6 and IL17 secretion through direct action on activated splenocytes.

In some embodiments, “preimplantation factor” or “PIF” may also refer to synthetic PIF-1, which replicates the native peptide's effect and exerts potent immune modulatory effects on activated peripheral blood mononuclear cell (PBMC) proliferation and cytokine secretion, acting through novel sites on PBMCs and having an effect which is distinct from known immunosuppressive drugs. In some embodiments, “preimplantation factor” or “PIF” refers to an amino acid selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, mimetics thereof, and combinations thereof that are about 75, 80, 81, 82, 83, 84 85, 86, 87, 88, 89, 90, 91, 92, 93, 94, 95, 96, 97, 98, or 99% homologous to any such amino acid.

As used herein, the terms “prevention” and “preventing,” when referring to a trauma and stressor-related disorder or symptom, refers to a reduction in the risk or likelihood that a mammalian subject will develop the disorder, symptom, condition, or indicator after treatment according to the invention, or a reduction in the risk or likelihood that a mammalian subject will exhibit a recurrence of the disorder, symptom, condition, or indicator once a subject has been treated according to the invention and cured or restored to a normal state (e.g., placed in remission from a targeted disorder). As used herein, the terms “treatment” or “treating,” when referring to disorders, refers to inhibiting or reducing the progression, nature, or severity of the subject condition or delaying the onset of the condition.

The term “prodrug”, as used herein typically refers to a derivative of an active compound or drug that requires a transformation under the conditions of use, such as within the body, to release the active drug. Prodrugs are frequently, but not necessarily, pharmacologically inactive until converted into the active drug. Prodrugs are typically obtained by masking a functional group in the drug believed to be in part required for activity with a progroup to form a promoiety which undergoes a transformation, such as cleavage, under the specified conditions of use to release the functional group, and hence the active drug. The cleavage of the promoiety can proceed spontaneously, such as by way of a hydrolysis reaction, or it can be catalyzed or induced by another agent, such as by an enzyme, by light, by acid, or by a change of or exposure to a physical or environmental parameter, such as a change of temperature. A wide variety of progroups, as well as the resultant promoieties, suitable for masking functional groups in active drugs, e.g., PIF, to yield prodrugs, are useful targets. For example, a hydroxyl functional group can be masked as a sulfonate, ester or carbonate promoiety, which can be hydrolyzed in vivo to revert the hydroxyl group. An amino functional group can be masked as an amide, carbamate, imine, urea, phosphenyl, phosphoryl or sulfenyl promoiety, which can be hydrolyzed in vivo to revert the amino group. A carboxyl group can be masked as an ester (e.g., silyl esters and thioesters), amide or hydrazide promoiety, which can be hydrolyzed in vivo to revert the carboxyl group. Other examples of suitable progroups and their respective promoieties will be apparent to those of skill in the art.

As used herein, the term “subject,” “individual” or “patient,” used interchangeably, means any animal, including mammals, such as mice, rats, other rodents, rabbits, dogs, cats, swine, cattle, sheep, horses, or primates, such as humans. As used herein, the term “animal” includes, but is not limited to, humans and non-human vertebrates such as wild animals, rodents, such as rats, ferrets, and domesticated animals, and farm animals, such as horses, pigs, cows, sheep, goats. In some embodiments, the animal is a mammal. In some embodiments, the animal is a human. In some embodiments, the animal is a non-human mammal. As used herein, the term “mammal” means any animal in the class Mammalia such as rodent (i.e., a mouse, a rat, or a guinea pig), a monkey, a cat, a dog, a cow, a horse, a pig, or a human. In some embodiments, the mammal is a human.

As used herein, the term “sustained release vehicle, matrix, binder, or coating material” refers to any vehicle, matrix, binder, or coating material that effectively, significantly delays dissolution of the active compound in vitro, and/or delays, modifies, or extends delivery of the active compound into the blood stream (or other in vivo target site of activity) of a subject following administration (e.g., oral administration), in comparison to dissolution and/or delivery provided by an “immediate release” formulation, as described herein, of the same dosage amount of the active compound. Accordingly, the term “sustained release vehicle, matrix, binder, or coating material” as used herein is intended to include all such vehicles, matrices, binders and coating materials known in the art as “sustained release”, “delayed release”, “slow release”, “extended release”, “controlled release”, “modified release”, and “pulsatile release” vehicles, matrices, binders and coatings. As used herein, “sustained release” and “sustained delivery” are evinced by a sustained, delayed, extended, or modified, in vitro or in vivo dissolution rate, in vivo release and/or delivery rate, and/or in vivo pharmacokinetic value(s) or profile.

As used herein, the term “therapeutic” means an agent utilized to treat, combat, ameliorate, prevent or improve an unwanted condition or disease of a subject. In part, embodiments of the present disclosure are directed to treating, ameliorating, preventing or improving one or more motor neuron disease (MND), in particular amyotrophic lateral sclerosis (ALS), and other conditions as described herein.

The terms “treating” or “treatment” as used herein, and as are well understood in the art, mean an approach for obtaining beneficial or desired results, including clinical results. Beneficial or desired clinical results can include, but are not limited to, alleviation or amelioration of one or more symptoms or conditions, diminishment of extent of injury or disease, stabilizing (i.e. not worsening) the state of injury or disease, delaying or slowing of injury or disease progression, amelioration or palliation of the injury or disease state, diminishment of the reoccurrence of injury or disease, and remission (whether partial or total), whether detectable or undetectable. Treatment methods optionally comprise administering to a subject a therapeutically effective amount of a composition and optionally consists of a single administration, or alternatively comprise a series of applications. The length of the treatment period depends on a variety of factors, such as the severity of the condition, the age of the patient, the concentration of the composition and components of the composition, the activity of the compositions and components of the composition, and/or a combination thereof. It will also be appreciated that the effective dosage of the composition and components of the composition used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions may be administered to the subject in an amount and for a duration sufficient to treat the patient. Treatment can also include eliciting a clinically significant response without excessive levels of side effects. Treatment also includes prolonging survival as compared to expected survival if not receiving treatment. For example, “treatment of a motor neuron disease” means an activity that alleviates or ameliorates any of the primary phenomena or secondary symptoms associated with one or more motor neuron disease (MND) and in particular amyotrophic lateral sclerosis (ALS).

### Description

Before the present compositions and methods are described, it is to be understood that this disclosure is not limited to the particular molecules, compositions, methodologies or protocols described, as these may vary. It is also to be understood that the terminology used in the description is for the purpose of describing the particular versions or embodiments only and is not intended to limit the scope of the present disclosure and exclude equivalents. It is understood that these embodiments are not limited to the particular methodology, protocols, cell lines, vectors, and reagents described, as these may vary. It also is to be understood that the terminology used herein is for the purpose of describing particular embodiments only and is not intended to limit the scope of the present embodiments or claims. The compositions described herein may include D amino acids, L amino acids, a racemic backbone of D and L amino acids, or any mixture thereof at each residue. That is, at each position, the residue may be a D amino acid residue or a L-amino acid residue and each position can be independently D or L of each other position, unless context dictates otherwise.

Without being bound by any particular theory, the compounds described herein act as agonists of PIF-mediated signal transduction via the receptor or receptors of PIF. Thus, these compounds modulate signaling pathways that provide significant therapeutic benefit in the treatment of, but not limited to, one or more motor neuron disease (MND) and in particular amyotrophic lateral sclerosis (ALS). The compounds of the present disclosure may exist in unsolvated forms as well as solvated forms, including hydrated forms. The compounds of the present disclosure also are capable of forming both pharmaceutically acceptable salts, including but not limited to acid addition and/or base addition salts. Furthermore, compounds of the present disclosure may exist in various solid states including an amorphous form (non-crystalline form), and in the form of clathrates, prodrugs, polymorphs, bio-hydrolyzable esters, racemic mixtures, non-racemic mixtures, or as purified stereoisomers including, but not limited to, optically pure enantiomers and diastereomers. In general, all of these forms can be used as an alternative form to the free base or free acid forms of the compounds, as described above and are intended to be encompassed within the scope of the present disclosure.

In some embodiments, the compounds of the present disclosure can be administered, inter alia, as pharmaceutically acceptable salts, esters, amides or prodrugs. The term “salts” refers to inorganic and organic salts of compounds of the present disclosure. In some embodiments, salts of the compositions comprising either a PIF or PIF analog or PIF mutant may be formed by reacting the free base, or a salt, enantiomer or racemate thereof, with one or more equivalents of the appropriate acid. In some embodiments, pharmaceutical acceptable salts of the present disclosure refer to analogs having at least one basic group or at least one basic radical. In some embodiments, pharmaceutical acceptable salts of the present disclosure comprise a free amino group, a free guanidino group, a pyrazinyl radical, or a pyridyl radical that forms acid addition salts. In some embodiments, the pharmaceutical acceptable salts of the present disclosure refer to analogs that are acid addition salts of the subject compounds with (for example) inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxybenzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid. When several basic groups are present mono- or poly-acid addition salts may be formed. The reaction may be carried out in a solvent or medium in which the salt is insoluble or in a solvent in which the salt is soluble, for example, water, dioxane, ethanol, tetrahydrofuran or diethyl ether, or a mixture of solvents, which may be removed in vacuo or by freeze drying. The reaction may also be a metathetical process or it may be carried out on an ion exchange resin. In some embodiments, the salts may be those that are physiologically tolerated by a patient. Salts according to the present disclosure may be found in their anhydrous form or as in hydrated crystalline form (i.e., complexed or crystallized with one or more molecules of water).

In one or more embodiments, a “therapeutically effective amount” or “effective amount” or “physiologically relevant amount” of a composition is an amount calculated to achieve a desired effect, i.e., to effectively inhibit or reduce symptoms and/or complications associated with one or more motor neuron disease (MND), in particular amyotrophic lateral sclerosis (ALS), or other conditions described herein. Effective amounts of compounds of the present disclosure can objectively or subjectively reduce or decrease the severity or frequency of symptoms associated with one or more motor neuron disease (MND), such as amyotrophic lateral sclerosis (ALS), or other conditions described herein.

The specific dose of a compound administered according to this disclosure to obtain therapeutic and/or prophylactic effects will, of course, be determined by the particular circumstances surrounding the case, including, for example, the compound administered, the route of administration, and the condition being treated. The compounds are effective over a wide dosage range and, for example, dosages per day will normally fall within the range of from about 0.01 mg/kg to about 10 mg/kg, about 0.1 mg/kg to about 1 mg/kg. In some embodiments, the therapeutically effective dose of PIF or PIF analog or peptide is about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, and 1 mg/kg.

It will be understood that the effective amount administered can also be determined by the physician in the light of the relevant circumstances including the condition to be treated, the choice of compound to be administered, and the chosen route of administration, and therefore the above dosage ranges are not intended to limit the scope of the disclosure in any way. A therapeutically effective amount of compound of this disclosure is typically an amount such that when it is administered in a physiologically tolerable excipient composition, it is sufficient to achieve an effective systemic concentration or local concentration in the tissue. In some embodiments, the term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed, the duration of the treatment; drugs used in combination or coincidentally with the specific compound employed; and like factors well known to the researcher, veterinarian, medical doctor or other clinician of ordinary skill.

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a co-therapy.

In one or more embodiments, the present invention relates to molecules, compositions and methods of treatment comprising same for treatment of a neurological disease, wherein the composition comprises an inventive molecule as described herein. The neurological disease is specifically ALS (amyotrophic lateral sclerosis) and its subtypes. ALS subtypes include bulbar-onset ALS and limb-onset ALS. In addition to ALS and its subtypes, optionally the inventive molecules could be used for treatment of other types of MND including primary lateral sclerosis (PLS), progressive bulbar palsy and progressive muscular atrophy, as described herein.

In one aspect the invention is a method for treating or preventing symptoms associated with amyotrophic lateral sclerosis (ALS) and related disorders.

As used herein, “conservative” amino acid substitutions may be defined as set out in Tables A, B, or C below. The PIF compounds of the disclosure include those wherein conservative substitutions (from either nucleic acid or amino acid sequences) have been introduced by modification of polynucleotides encoding polypeptides of the disclosure. Amino acids can be classified according to physical properties and contribution to secondary and tertiary protein structure. A conservative substitution is recognized in the art as a substitution of one amino acid for another amino acid that has similar properties. In some embodiments, the conservative substitution is recognized in the art as a substitution of one nucleic acid for another nucleic acid that leads to a conservative amino acid substitution. Exemplary conservative substitutions are set out in Table A.

TABLE A
Conservative Substitutions I

| Side Chain Characteristics | Amino Acid |
|---|---|
| Aliphatic | |
| Non-polar | G, A, P, I, L, V, F |
| Polar - uncharged | C, S, T, M, N, Q |
| Polar - charged | D, E, K, R |
| Aromatic | H, F, W, Y |
| Other | N, Q, D, E |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain Characteristics | Amino Acid |
|---|---|
| Non-polar (hydrophobic) | |
| Aliphatric | A, L, I, V, P |
| Aromatic | F, W, Y |
| Sulfur-containing | M |
| Borderline | G, Y |
| Uncharged-polar | |
| Hydroxyl | S, T, Y |
| Amides | N, Q |
| Sulfhydryl | C |
| Borderline | C: G, Y |
| Positively Charged (Basic) | K, R, H |
| Negatively Charged (Acidic) | D, E |

Alternately, exemplary conservative substitutions are set out in Table C.

TABLE C
Conservative Substitutions III

| Original Residue | Exemplary Substitution |
|---|---|
| Ala (A) | Val, Leu, Ile, Met |
| Arg (R) | Lys, His |
| Asn (N) | Gln |
| Asp (D) | Glu |
| Cys (C) | Ser, Thr |
| Gln (Q) | Asn |
| Glu (E) | Asp |
| Gly (G) | Ala, Val, Leu, Pro |
| His (H) | Lys, Arg Ile (I) Leu, Val, Met, Ala, |
| | Phe |
| Leu (L) | Ile, Val, Met, Ala, Phe |
| Lys (K) | Arg, His |
| Met (M) | Leu, Ile, Val, Ala |
| Phe (F) | Trp, Tyr, Ile |
| Pro (P) | Gly, Ala, Val, Leu, Ile |
| Ser (S) | Thr |
| Thr (T) | Ser |
| Trp (W) | Tyr, Phe, Ile |
| Tyr (Y) | Trp, Phe, Thr, Ser |
| Val (V) | Ile, Leu, Met, Ala |

As used herein, the terms “peptide,” “polypeptide” and “protein” are used interchangeably and refer to two or more amino acids covalently linked by an amide bond or non-amide equivalent. The peptides of the disclosure can be of any length. For example, the peptides can have from about two to about 100 or more residues, such as, 5 to 12, 12 to 15, 15 to 18, 18 to 25, 25 to 50, 50 to 75, 75 to 100, or more in length. Preferably, peptides are from about 2 to about 18 residues in length. The peptides of the disclosure also include 1- and d-isomers, and combinations of 1- and d-isomers. The peptides can include modifications typically associated with posttranslational processing of proteins, for example, cyclization (e.g., disulfide or amide bond), phosphorylation, glycosylation, carboxylation, ubiquitination, myristylation, or lipidation. In some embodiments, the compositions or pharmaceutical compositions of the disclosure relate to analogs of any PIF sequence set forth in Table 1 that share no less than about 70%, about 75%, about 79%, about 80%, about 85%, about 86%, about 87%, about 88%, about 89%, about 90%, about 91%, about 92%, about 93%, about 94% about 95%, about 96%, about 97%, about 98%, about 99% homology with any one or combination of PIF sequences set forth in Table 1. In some embodiments, PIF may refer to an amino acid sequence selected from SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 or a functional fragment thereof that is about 70%, 75%, 80%, 85%, 90%, 91%, 92%, 93%, 94%, 95%, 96%, 97%, 98%, or 99% homologous to any such amino acid sequence. In some embodiments, PIF may refer to an amino acid sequence comprising, consisting essentially of, or consisting of a sequence that is at least 70%, 75%, 80%, 85%, 86%, 87%, 90%, 91%, 92%, 93%, 94%, 95%, 96%, 97%, 98%, or 99% homologous to SEQ ID. NO: 20.

In some embodiments, the PIF mutant comprises a sequence selected from: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39 and SEQ ID NO: 40.

In some embodiments, the PIF mutant comprises a sequence selected from: XVZIKPGSANKPSD (SEQ ID NO: 21), XVZIKPGSANKPS (SEQ ID NO: 22), XVZIKPGSANKP (SEQ ID NO: 23), XVZIKPGSANK (SEQ ID NO: 24), XVZIKPGSAN (SEQ ID NO: 25), XVZIKPGSA (SEQ ID NO: 26), XVZIKPGS (SEQ ID NO: 27), XVZIKPG (SEQ ID NO: 28), XVZIKP (SEQ ID NO: 29), XVZIK (SEQ ID NO: 30), XVZI (SEQ ID NO: 31), or XVZ wherein X is a non-natural amino acid or a naturally occurring amino acid. In some embodiments, the PIF mutant comprises a sequence selected from: XVZIKPGSANKPSD (SEQ ID NO: 21), XVZIKPGSANKPS (SEQ ID NO: 22), XVZIKPGSANKP (SEQ ID NO: 23), XVZIKPGSANK (SEQ ID NO: 24), XVZIKPGSAN (SEQ ID NO: 25), XVZIKPGSA (SEQ ID NO: 26), XVZIKPGS (SEQ ID NO: 27), XVZIKPG (SEQ ID NO: 28), XVZIKP (SEQ ID NO: 29), XVZIK (SEQ ID NO: 30), XVZI (SEQ ID NO: 31), or XVZ wherein X is a non-natural amino acid or a naturally occurring amino acid except that X is not methionine if Z is arginine, and Z is not arginine if X is methionine. In some embodiments, the PIF analog or mutant is synthetic or synthetically made.

Peptides disclosed herein further include compounds having amino acid structural and functional analogs, for example, peptidomimetics having synthetic or non-natural amino acids (such as a norleucine) or amino acid analogues or non-natural side chains, so long as the mimetic shares one or more functions or activities of compounds of the disclosure. The compounds of the disclosure therefore include “mimetic” and “peptidomimetic” forms. As used herein, a “non-natural side chain” is a modified or synthetic chain of atoms joined by covalent bond to the alpha-carbon atom, beta-carbon atom, or gamma-carbon atom which does not make up the backbone of the polypeptide chain of amino acids. The peptide analogs may comprise one or a combination of non-natural amino-acids chosen from: norvaline, tert-butylglycine, phenylglycine, He, 7-azatryptophan, 4-fluorophenylalanine, N-methyl-methionine, N-methyl-valine, N-methyl-alanine, sarcosine, N-methyl-tert-butylglycine, N-methyl-leucine, N-methyl-phenylglycine, N-methyl-isoleucine, N-methyl-tryptophan, N-methyl-7-azatryptophan, N-methyl-phenylalanine, N-methyl-4-fluorophenylalanine, N-methyl-threonine, N-methyl-tyrosine, N-methyl-valine, N-methyl-lysine, homocysteine. Non-natural side chains are disclosed in the art in the following publications: WO/2013/172954, WO2013123267, WO/2014/071241, WO/2014/138429, WO/2013/050615, WO/2013/050616, WO/2012/166559, U.S. Application No. 20150094457, Ma, Z., and Hartman, M. C. (2012). In Vitro Selection of Unnatural Cyclic Peptide Libraries via mRNA Display. In J. A. Douthwaite & R. H. Jackson (Eds.), Ribosome Display and Related Technologies: Methods and Protocols (pp. 367-390). Springer New York., all of which are incorporated by reference in their entireties.

The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics, as further described below. The term “analog” refers to any polypeptide comprising at least one alpha-amino acid and at least one non-native amino acid residue, wherein the polypeptide is structurally similar to a naturally occurring full-length PIF protein and shares the biochemical or biological activity of the naturally occurring full-length protein upon which the analog is based. In some embodiments, the compositions, pharmaceutical compositions and kits comprise a peptide or peptidomimetic sharing share no less than about 70%, about 75%, about 79%, about 80%, about 85%, about 86%, about 87%, about 90%, about 93%, about 94% about 95%, about 96%, about 97%, about 98%, about 99% homology with any one or combination of PIF sequences set forth in Table 1; and wherein one or a plurality of amino acid residues is a non-natural amino acid residue or an amino acid residue with a non-natural sidechain. In some embodiments, peptide or peptide mimetics are provided, wherein a loop is formed between two cysteine residues. In some embodiments, the peptidomimetic may have many similarities to natural peptides, such as: amino acid side chains that are not found among the known 20 proteinogenic amino acids, non-peptide-based linkers used to effect cyclization between the ends or internal portions of the molecule, substitutions of the amide bond hydrogen moiety by methyl groups (N-methylation) or other alkyl groups, replacement of a peptide bond with a chemical group or bond that is resistant to chemical or enzymatic treatments, N- and C-terminal modifications, and conjugation with a non-peptidic extension (such as polyethylene glycol, lipids, carbohydrates, nucleosides, nucleotides, nucleoside bases, various small molecules, or phosphate or sulfate groups). As used herein, the term “cyclic peptide mimetic” or “cyclic polypeptide mimetic” refers to a peptide mimetic that has as part of its structure one or more cyclic features such as a loop, bridging moiety, and/or an internal linkage.

In some embodiments, peptide or peptide mimetics are provided, wherein the loop comprises a bridging moiety selected from the group consisting of:

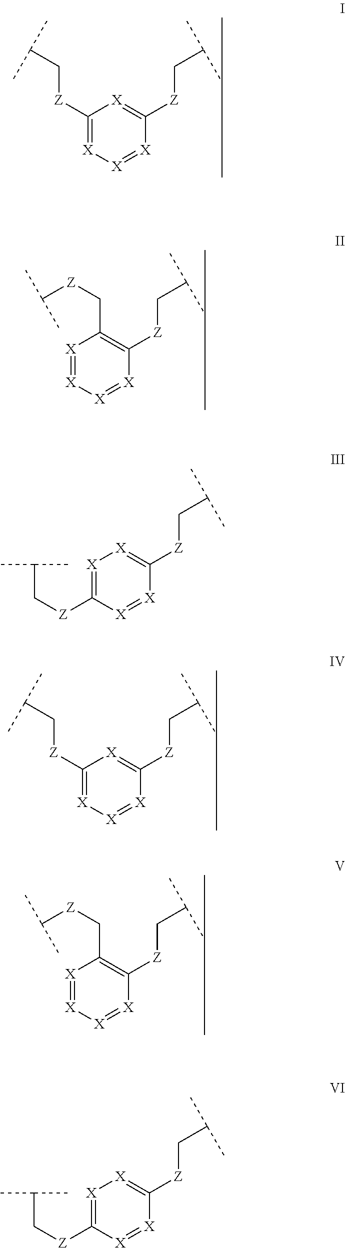

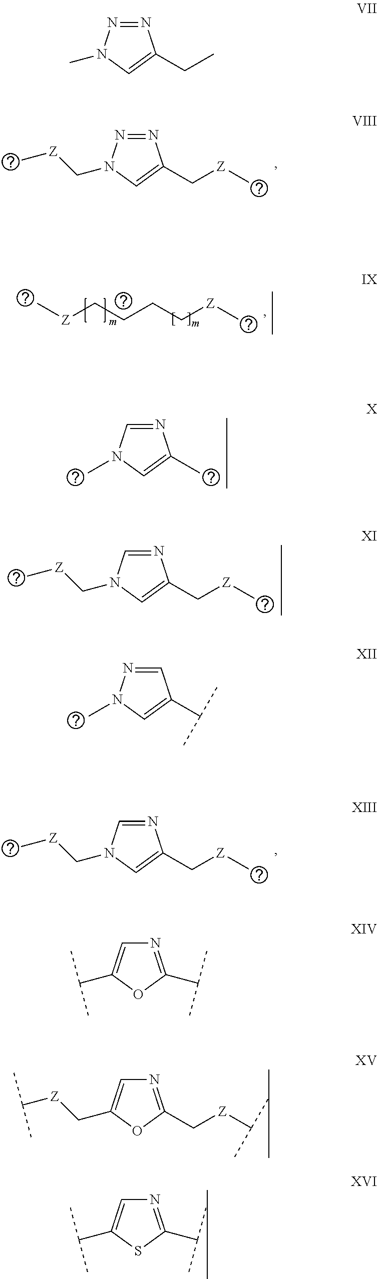

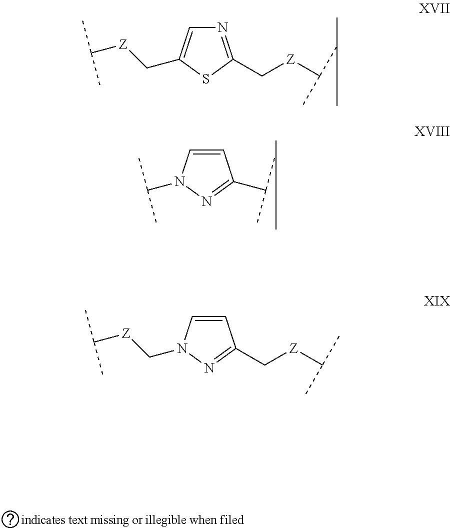

wherein each X is independently N or CH, such that no ring contains more than 2 N; each Z is independently a bond, NR, O, S, CH2, C(O)NR, NRC(O), S(O)vNR, NRS(O)v; each m is independently selected from 0, 1, 2, and 3; each v is independently selected from 1 and 2; each R is independently selected from H and C1-C6; and each bridging moiety is connected to the peptide by independently selected C0-C6 spacers.

In some embodiments, the PIF peptides of the disclosure are modified to produce peptide mimetics by replacement of one or more naturally occurring side chains of the 20 genetically encoded amino acids (or D-amino acids) with other side chains, for instance with groups such as alkyl, lower alkyl, cyclic 4-, 5-, 6-, to 7 membered alkyl, amide, amide lower alkyl, amide di (lower alkyl), lower alkoxy, hydroxy, carboxy and the lower ester derivatives thereof, and with 4-, 5-, 6-, to 7 membered heterocyclics. For example, proline analogs can be made in which the ring size of the proline residue is changed from 5 members to 4, 6, or 7 members. Cyclic groups can be saturated or unsaturated, and if unsaturated, can be aromatic or nonaromatic. Heterocyclic groups can contain one or more nitrogen, oxygen, and/or sulphur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl. Peptidomimetics may also have amino acid residues that have been chemically modified by phosphorylation, sulfonation, biotinylation, or the addition or removal of other moieties.

In a further embodiment a compound of the formula R1—R2—R3—R4—R5—R6—R7—R8—R-9—R10—R11—R12—R13—R14—R15, wherein R1 is Met or a mimetic of Met, R2 is Val or a mimetic of Val, R3 is Arg or a mimetic of Arg, or any amino acid, R4 is Ile or a mimetic of Ile, R5 is Lys or a mimetic of Lys, R6 is Pro or a mimetic of Pro, R7 is Gly or a mimetic of Gly, R8 is Ser or a mimetic of Ser, R9 is Ala or a mimetic of Ala, R10 is Asn or a mimetic of Asn, R11 is Lys or a mimetic of Lys, R12 is Pro or a mimetic of Pro, R13 is Ser or a mimetic of Ser, R14 is Asp or a mimetic of Asp and R15 is Asp or a mimetic of Asp is provided. In a further embodiment, a compound comprising the formula R1—R2—R3—R4—R5—R6—R7—R8—R-9—R10, wherein R1 is Ser or a mimetic of Ser, R2 is Gln or a mimetic of Gln, R3 is Ala or a mimetic of Ala, R4 is Val or a mimetic of Val, R5 is Gln or a mimetic of Gln, R6 is Glu or a mimetic of Glu, R7 is His or a mimetic of His, R is Ala or a mimetic of Ala, R9 is Ser or a mimetic of Ser, and R10 is Thr or a mimetic of Thr; a compound comprising the formula R1—R2—R3—R4—R5—R6—R7—R8—R-9—R10—R11—R12—R13—R14—R15—R-16—R17—R18, wherein R1 is Ser or a mimetic of Ser, R2 is Gly or a mimetic of Gly, R3 is Ile or a mimetic of Ile, R4 is Val or a mimetic of Val, R5 is Ile or a mimetic of Ile, R6 is Tyr or a mimetic of Tyr, R7 is Gln or a mimetic of Gln, R8 is Tyr or a mimetic of Tyr, R9 is Met or a mimetic of Met, R10 is Asp or a mimetic of Asp, R11 is Asp or a mimetic of Asp, R12 is Arg or a mimetic of Arg, R13 is Tyr or a mimetic of Tyr, R14 is Val or a mimetic of Val, R15 is Gly or a mimetic of Gly, R16 is Ser or a mimetic of Ser, R17 is Asp or a mimetic of Asp and R18 is Leu or a mimetic of Leu; and a compound comprising the formula R1—R2—R3—R4—R5—R6—R7—R.-sub.8—R9, wherein R1 is Val or a mimetic of Val, R2 is Ile or a mimetic of Ile, R3 is Ile or a mimetic of Ile, R4 is Ile or a mimetic of Ile, R5 is Ala or a mimetic of Ala, R6 is Gln or a mimetic of Gln, R7 is Tyr or a mimetic of Tyr, R8 is Met or a mimetic of Met, and R9 is Asp or a mimetic of Asp is provided. In some embodiments, R3 is not Arg or a mimetic of Arg.

A variety of techniques are available for constructing peptide mimetics with the same or similar desired biological activity as the corresponding native but with more favorable activity than the peptide with respect to solubility, stability, and/or susceptibility to hydrolysis or proteolysis (see, e.g., Morgan & Gainor, Ann. Rep. Med. Chern. 24, 243-252, 1989). Certain peptidomimetic compounds are based upon the amino acid sequence of the peptides of the disclosure. Often, peptidomimetic compounds are synthetic compounds having a three-dimensional structure (i.e., a “peptide motif”) based upon the three-dimensional structure of a selected peptide. The peptide motif provides the peptidomimetic compound with the desired biological activity, i.e., binding to PIF receptors, wherein the binding activity of the mimetic compound is not substantially reduced and is often the same as or greater than the activity of the native peptide on which the mimetic is modeled. Peptidomimetic compounds can have additional characteristics that enhance their therapeutic application, such as increased cell permeability, greater affinity and/or avidity and prolonged biological half-life.

Peptidomimetic design strategies are readily available in the art (see, e.g., Ripka & Rich, Curr. Op. Chern. Bioi. 2, 441-452, 1998; Hruby et al., Curr. Op. Chem. Bioi. 1, 114-119, 1997; Hruby & Baise, Curr. Med. Chern. 9, 945-970, 2000). One class of peptidomimetics a backbone that is partially or completely non-peptide but mimics the peptide backbone atom—for atom and comprises side groups that likewise mimic the functionality of the side groups of the native amino acid residues. Several types of chemical bonds, e.g., ester, thioester, thioamide, retroamide, reduced carbonyl, dimethylene and ketomethylene bonds, are known in the art to be generally useful substitutes for peptide bonds in the construction of protease-resistant peptidomimetics. Another class of peptidomimetics comprises a small non-peptide molecule that binds to another peptide or protein, but which is not necessarily a structural mimetic of the native peptide. Yet another class of peptidomimetics has arisen from combinatorial chemistry and the generation of massive chemical libraries. These generally comprise novel templates which, though structurally unrelated to the native peptide, possess necessary functional groups positioned on a nonpeptide scaffold to serve as “topographical” mimetics of the original peptide (Ripka & Rich, 1998, supra).

The first natural PIF compound identified, termed nPIF (SEQ ID NO: 1), is a 15 amino acid peptide. A synthetic version of this peptide, sPIF (SEQ ID NO:13), showed activity that was similar to the native peptide, nPIF (SEQ ID NO: I). This peptide is homologous to a small region of the Circumsporozoite protein, a malaria parasite. The second PIF peptide (SEQ ID NO: 7), includes 13 amino acids and shares homology with a short portion of a large protein named thyroid and retinoic acid transcription co-repressor, which is identified as a receptor-interacting factor, (SMRT); the synthetic version is sPIF-2 (SEQ ID NO:14). The third distinct peptide, nPIF-3 (SEQ ID NO:10), consists of 18 amino acids and matches a small portion of reverse transcriptase; the synthetic version of this peptide sPIF-3 is (SEQ ID NO:15). nPIF-4 (SEQ ID NO: 12) shares homology with a small portion of reverse transcriptase.

A list of PIF peptides, both natural and synthetic, are provided below in Table 1. Antibodies to various PIF peptides and scrambled PIF peptides are also provided.

TABLE 1
PIF Peptides

| (SEQ ID NO) | Peptide | Amino Acid Sequence |
|---|---|---|
| SEQ ID NO: 1 | nPIF-115 | MVRIKPGSANKPSDD |
| isolated native, matches | | |
| region of Circumsporozoite | | |
| protein (Malaria) | | |
| SEQ ID NO: 2 | nPIF-1(15-alter) | MVRIKYGSYNNKPSD |
| isolated native, matches | | |
| region of Circumsporozoite | | |
| protein (Malaria) | | |
| SEQ ID NO: 3 | nPIF-1(13) | MVRIKPGSANKPS |
| isolated native, matches | | |
| region of Circumsporozoite | | |
| protein (Malaria) | | |
| SEQ ID NO: 4 | nPIF-1(9) | MVRIKPGSA |
| isolated native, matches | | |
| region of Circumsporozoite | | |
| protein (Malaria) | | |
| SEQ ID NO: 5 | scrPIF-115 | GRVDPSNKSMPKDIA |
| synthetic, scrambled amino | | |
| acid sequence from region of | | |
| Circumsporozoite protein | | |
| Malaria | | |
| SEQ ID NO: 6 | nPIF-2(10) | SQAVQEHAST |
| isolated native, matches | | |
| region of human retinoid and | | |
| thyroid hormone receptor- | | |
| SMRT | | |
| SEQ ID NO: 7 | nPIF-2(13) | SQAVQEHASTNMG |
| isolated native, matches | | |
| region of human retinoid and | | |
| thyroid hormone receptor | | |
| (SMRT) | | |
| SEQ ID NO: 8 | scrPIF-2(13) | EVAQHSQASTMNG |
| synthetic, scrambled amino | | |
| acid sequence from region of | | |
| human retinoid and thyroid | | |
| hormone receptor SMRT | | |
| SEQ ID NO: 9 | scrPIF-2(14) | GQASSAQMNSTGVH |
| SEQ ID NO: 10 | nPIF-3(18) | SGIVIYQYMDDRYVGSDL |
| isolated native, matches | | |
| region of Rev Trans | | |
| SEQ ID NO: 11 | Neg control for | GMRELQRSANK |
| synthetic, scrambled amino | negPIF- | |
| acid sequence from region of | 1(15) | |
| Circumsporozoite protein | | |
| Malaria | | |
| SEQ ID NO: 12 | nPIF-4(9) | VIIIAGin YMD |
| isolated native, matches | | |
| region of Rev Trans antibody | | |
| of native isolated nPIF-115 | | |
| AbPIF-1(15) | | |
| SEQ ID NO: 13 | SPIF-1(15) | MVRIKPGSANKPSDD |
| synthetic, amino acid | | |
| sequence from region of | | |
| Circumsporozoite protein | | |
| Malaria | | |
| SEQ ID NO: 14 | SPIF-2(13) | SQAVQEHASTNMG |
| synthetic, amino acid | | |
| sequence from of human | | |
| retinoid and thyroid hormone | | |
| receptor SMRT | | |
| SEQ ID NO: 15 | SPIF-3(18) | SGIVIYQYMDDRYVGSDL |
| synthetic, amino acid | | |
| sequence from region of | | |
| Circumsporozoite | protein | |
| Malaria | | |
| SEQ ID NO: 16 | SPIF-1(9) | MVRIKPGSA |
| synthetic, amino acid of | | |
| sequence from region of | | |
| Circumsporozoite Malaria | | |
| SEQ ID NO: 17 | SP1F-4(9) | VIIIAQYMD |
| Synthetic | | |
| SEQ ID NO: 18 | SP1F-1(5) | MVRIK |
| Synthetic | | |
| SEQ ID NO: 19 | SP1F-1(4) | PGSA |
| Synthetic | | |
| SEQ ID NO: 20 | PIF (−3) | MVXIKPGSANKPSDD |
| Synthetic | | |

n = native, s = synthetic, scr = scrambled, same AA, ( ) = number of AA, Ab =
antibody, X = any amino acid, except arginine

In some embodiments of the present disclosure, a PIF peptide is provided. Such PIF peptides may be useful for treating one or more motor neuron disease (MND), such as amyotrophic lateral sclerosis (ALS), or for any other condition described herein. In some embodiments, the PIF peptides can be used to treat the autoimmune conditions described herein. In some embodiments, the PIF peptides can be used to treat paralysis, such as what is seen in multiple sclerosis (“MS”). Accordingly, in some embodiments, methods of treating MS induced paralysis are provided, wherein the method comprises administering a PIF peptide, such as SEQ ID NO: 13 to the subject with MS induced In some embodiments, the PIF peptides can be used to treat the autoimmune conditions described herein. In some embodiments, the paralysis is inflammation induced paralysis. In some embodiments, the inflammation is localized to the CNS or peripheral nervous system.

In another embodiment, a pharmaceutical composition comprising a PIF peptide is provided. In some embodiments, the pharmaceutical composition comprises a therapeutically effective amount of a PIF peptide or a pharmaceutically acceptable salt thereof.

In some embodiments, a method of treating ALS is provided. In some embodiments, the method comprises administering an effective amount of a PIF peptide to a subject in need thereof.

In some embodiments, a method for treating ALS comprising administering an effective amount of a PIF peptide in combination with one or more immunotherapeutic, anti-epileptic, diuretic, or blood pressure controlling drugs or compounds to a subject in need thereof is provided. Such a combination may enhance the effectiveness of the treatment of either component alone or may provide less side effects and/or enable a lower dose of either component.

Ultimately, a novel embryo-derived peptide, PIF, creates a tolerogenic state at low doses following short-term treatment leading to long-term protection in several distinct severe autoimmune models. This effect is exerted without apparent toxicity.

For therapeutic treatment of the specified indications, a PIF peptide may be administered as such, or can be compounded and formulated into pharmaceutical compositions in unit dosage form for parenteral, transdermal, rectal, nasal, local intravenous administration, or oral administration. In some embodiments, it is administered subcutaneously. Such pharmaceutical compositions are prepared in a manner well known in the art and comprise at least one active PIF peptide associated with a pharmaceutically carrier. The term “active compound”, as used throughout this specification, refers to at least one compound selected from compounds of the formulas or pharmaceutically acceptable salts thereof.

In such a composition, the active compound is known as “active ingredient.” In making the compositions, the active ingredient can be mixed with a carrier, or diluted by a carrier, or enclosed within a carrier that may be in the form of a capsule, sachet, paper or other container. When the carrier serves as a diluent, it may be a solid, semisolid, or liquid material that acts as a vehicle, excipient of medium for the active ingredient. Thus, the composition can be in the form of tablets, pills, powders, lozenges, sachets, cachets, elixirs, emulsion, solutions, syrups, suspensions, soft and hard gelatin capsules, sterile injectable solutions, and sterile packaged powders.

The terms “pharmaceutical preparation” or “pharmaceutical composition” includes preparations suitable for administration to mammals, e.g., humans. When the compounds of the present disclosure are administered as pharmaceuticals to mammals, e.g., humans, they can be given per se or as a pharmaceutical composition containing, for example, from about 0.1 to about 99.5% of active ingredient in combination with a pharmaceutically acceptable carrier.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The phrase “pharmaceutically acceptable carrier” is art recognized and includes a pharmaceutically acceptable material, composition or vehicle, suitable for administering compounds of the present disclosure to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar, buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in “Remington's Pharmaceutical Sciences” by E. W. Martin, which is incorporated herein by reference in its entirety. In some embodiments, the pharmaceutically acceptable carrier is sterile and pyrogen-free water. In some embodiments, the pharmaceutically acceptable carrier is Ringer's Lactate, sometimes known as lactated Ringer's solution.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present disclosure include those suitable for oral, nasal, topical, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Some examples of suitable carriers, excipients, and diluents include lactose, dextrose, sucrose, sorbitol, mannitol, starches, gum acacia, calcium phosphate alginates, calcium salicate, microcrystalline cellulose, polyvinylpyrrolidone, cellulose, tragacanth, gelatin, syrup, methyl cellulose, methyl- and propylhydroxybenzoates, tale, magnesium stearate, water, and mineral oil. The formulations can additionally include lubricating agents, wetting agents, emulsifying and suspending agents, preserving agents, sweetening agents or flavoring agents. The compositions may be formulated so as to provide quick, sustained, or delayed release of the active ingredient after administration to the patient by employing procedures well known in the art.

For oral administration, a compound can be admixed with carriers and diluents, molded into tablets, or enclosed in gelatin capsules. The mixtures can alternatively be dissolved in liquids such as 10% aqueous glucose solution, isotonic saline, sterile water, or the like, and administered intravenously or by injection.

The local delivery of inhibitory amounts of active compound for the treatment of immune disorders can be by a variety of techniques that administer the compound at or near the targeted site. Examples of local delivery techniques are not intended to be limiting but to be illustrative of the techniques available. Examples include local delivery catheters, site specific carriers, implants, direct injection, or direct applications, such as topical application.

Local delivery by an implant describes the surgical placement of a matrix that contains the pharmaceutical agent into the affected site. The implanted matrix releases the pharmaceutical agent by diffusion, chemical reaction, or solvent activators.

For example, in some aspects, the disclosure is directed to a pharmaceutical composition comprising a PIF peptide, and a pharmaceutically acceptable carrier or diluent, or an effective amount of pharmaceutical composition comprising a PIF peptide.

The compounds of the present disclosure can be administered in the conventional manner by any route where they are active. Administration can be systemic, topical, or oral. For example, administration can be, but is not limited to, parenteral, subcutaneous, intravenous, intramuscular, intraperitoneal, transdermal, oral, buccal, ocular routes, intravaginally, by inhalation, by depot injections, or by implants. Thus, modes of administration for the compounds of the present disclosure (either alone or in combination with other pharmaceuticals) can be, but are not limited to, subligual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered will depend on the characteristics of the subject being treated, e.g., the particular mammal or human treated, age, weight, health, types of concurrent treatment, if any, and frequency of treatments, and can be easily determined by one of skill in the art (e.g., by the clinician).

Pharmaceutical formulations containing the compounds of the present disclosure and a suitable carrier can be solid dosage forms which include, but are not limited to, tablets, capsules, cachets, pellets, pills, powders and granules; topical dosage forms which include, but are not limned to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present disclosure. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compounds of the present disclosure can be formulated for parenteral administration by injection, e.g., by bolus injection or continuous infusion. The compounds can be administered by continuous infusion subcutaneously over a predetermined period of time. Formulations for injection can be presented in unit dosage form, e.g., in ampoules or in multi-dose containers, with an added preservative. The compositions can take such forms as suspensions, solutions, or emulsions in oily or aqueous vehicles, and can contain formulatory agents such as suspending, stabilizing and/or dispersing agents.

For oral administration, the compounds can be formulated readily by combining these compounds with pharmaceutically acceptable carriers well known in the art. Such carriers enable the compounds of the disclosure to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, alter adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragecanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, scaled capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compounds for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds of the present disclosure can also be formulated in rectal compositions such as suppositories or retention enemas, e.g., containing conventional suppository bases such as cocoa butter or other glycerides.

In addition to the formulations described previously, the compounds of the present disclosure can also be formulated as a depot preparation. Such long-acting formulations can be administered by implantation (for example subcutaneously or intramuscularly) or by intramuscular injection.

Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds of the present disclosure, for example, can be applied to a plaster, or can be applied by transdermal, therapeutic systems that are consequently supplied to the organism.

Pharmaceutical compositions of the compounds also can comprise suitable solid or gel phase carriers or excipients. Examples of such carriers or excipients include but are not limited to calcium carbonate, calcium phosphate, various sugars, starches, cellulose derivates, gelatin, and polymers such as, e.g., polyethylene glycols.

For parenteral administration, analog can be, for example, formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques. For example, a parenteral composition suitable for administration by injection is prepared by dissolving 1.5% by weight of analog in 0.9% sodium chloride solution.

The present invention relates to routes of administration include intramuscular, sublingual, intravenous, intraperitoneal, intrathecal, intravaginal, intraurethral, intradermal, intrabuccal, via inhalation, via nebulizer and via subcutaneous injection. Alternatively, the pharmaceutical composition may be introduced by various means into cells that are removed from the individual. Such means include, for example, microprojectile bombardment and liposome or other nanoparticle device.

Solid dosage forms for oral administration include capsules, tablets, pills, powders and granules. In solid dosage forms, the analogs are generally admixed with at least one inert pharmaceutically acceptable carrier such as sucrose, lactose, starch, or other generally regarded as safe (GRAS) additives. Such dosage forms can also comprise, as is normal practice, an additional substance other than an inert diluent, e.g., lubricating agent such as magnesium state. With capsules, tablets, and pills, the dosage forms may also comprise a buffering agent. Tablets and pills can additionally be prepared with enteric coatings, or in a controlled release form, using techniques know in the art.

Liquid dosage forms for oral administration include pharmaceutically acceptable emulsions, solutions, suspensions and syrups, with the elixirs containing an inert diluent commonly used in the art, such as water. These compositions can also include one or more adjuvants, such as wetting agent, an emulsifying agent, a suspending agent, a sweetening agent, a flavoring agent or a perfuming agent.

In another embodiment of the invention the composition of the invention is used to treat a patient suffering from, or susceptible to Type I adult or juvenile diabetes, multiple sclerosis, Crohn's, or autoimmune hepatitis.

One of skill in the art will recognize that the appropriate dosage of the compositions and pharmaceutical compositions may vary depending on the individual being treated and the purpose. For example, the age, body weight, and medical history of the individual patient may affect the therapeutic efficacy of the therapy. Further, a lower dosage of the composition may be needed to produce a transient cessation of symptoms, while a larger dose may be needed to produce a complete cessation of symptoms associated with the disease, disorder, or indication. A competent physician can consider these factors and adjust the dosing regimen to ensure the dose is achieving the desired therapeutic outcome without undue experimentation. It is also noted that the clinician and/or treating physician will know how and when to interrupt, adjust, and/or terminate therapy in conjunction with individual patient response. Dosages may also depend on the strength of the particular analog chosen for the pharmaceutical composition.

The dose of the composition or pharmaceutical compositions may vary. The dose of the composition may be once per day. In some embodiments, multiple doses may be administered to the subject per day. In some embodiments, the total dosage is administered in at least two application periods. In some embodiments, the period can be an hour, a day, a month, a year, a week, or a two-week period. In an additional embodiment of the invention, the total dosage is administered in two or more separate application periods, or separate doses over the course of an hour, a day, a month, a year, a week, or a two-week period.

In some embodiments, subjects can be administered the composition in which the composition is provided in a daily dose range of about 0.0001 mg/kg to about 5000 mg/kg of the weight of the subject. The dose administered to the subject can also be measured in terms of total amount of PIF peptide or PIF analog or pharmaceutically acceptable salt thereof administered per day. In some embodiments, a subject is administered from about 0.001 to about 3000 milligrams of PIF peptide or PIF analog or pharmaceutically acceptable salt thereof per day. In some embodiments, a subject is administered up to about 2000 milligrams of PIF peptide or PIF analog or pharmaceutically acceptable salt thereof per day. In some embodiments, a subject is administered up to about 1800 milligrams of PIF peptide or PIF analog or pharmaceutically acceptable salt thereof per day. In some embodiments, a subject is administered up to about 1600 milligrams of PIF peptide or PIF analog or pharmaceutically acceptable salt thereof per day. In some embodiments, a subject is administered up to about 1400 milligrams of PIF peptide or PIF analog or pharmaceutically acceptable salt thereof per day. In some embodiments, a subject is administered up to about 1200 milligrams of PIF peptide or PIF analog or pharmaceutically acceptable salt thereof per day. In some embodiments, a subject is administered up to about 1000 milligrams of PIF peptide or PIF analog or pharmaceutically acceptable salt thereof per day. In some embodiments, a subject is administered up to about 800 milligrams of PIF peptide or PIF analog or pharmaceutically acceptable salt thereof per day. In some embodiments, a subject is administered from about 0.001 milligrams to about 700 milligrams of PIF peptide or PIF analog or pharmaceutically acceptable salt thereof per dose. In some embodiments, a subject is administered up to about 700 milligrams of PIF peptide or PIF analog per dose. In some embodiments, a subject is administered up to about 600 milligrams of PIF peptide or PIF analog or pharmaceutically acceptable salt thereof per dose. In some embodiments, a subject is administered up to about 500 milligrams of PIF peptide or PIF analog or pharmaceutically acceptable salt thereof per dose. In some embodiments, a subject is administered up to about 400 milligrams of PIF peptide or PIF analog or pharmaceutically acceptable salt thereof per dose. In some embodiments, a subject is administered up to about 300 milligrams of PIF peptide or PIF analog or pharmaceutically acceptable salt thereof per dose. In some embodiments, a subject is administered up to about 200 milligrams of PIF peptide or PIF analog or pharmaceutically acceptable salt thereof per dose. In some embodiments, a subject is administered up to about 100 milligrams of PIF peptide or PIF analog or pharmaceutically acceptable salt thereof per dose. In some embodiments, a subject is administered up to about 50 milligrams of PIF peptide or PIF analog or pharmaceutically acceptable salt thereof per dose.

In some embodiments, subjects can be administered the composition in which the composition comprising a PIF peptide or PIF analog or pharmaceutically acceptable salt thereof is administered in a daily dose range of about 0.0001 mg/kg to about 5000 mg/kg of the weight of the subject. In some embodiments, the composition comprising a PIF analog or pharmaceutically acceptable salt thereof is administered in a daily dosage of up to about 450 mg/kg of the weight of the subject. In some embodiments, the composition comprising a PIF peptide or PIF analog or pharmaceutically acceptable salt thereof is administered in a daily dosage of up to about 400 mg/kg of the weight of the subject. In some embodiments, the composition comprising a PIF peptide or PIF analog or pharmaceutically acceptable salt thereof is administered in a daily dosage of up to about 350 mg/kg of the weight of the subject. In some embodiments, the composition comprising a PIF peptide or PIF analog or pharmaceutically acceptable salt thereof is administered in a daily dosage of up to about 300 mg/kg of the weight of the subject. In some embodiments, the composition comprising a PIF peptide or PIF analog or pharmaceutically acceptable salt thereof is administered in a daily dosage of up to about 250 mg/kg of the weight of the subject. In some embodiments, the composition comprising PIF peptide or a PIF analog or pharmaceutically acceptable salt thereof is administered in a daily dosage of up to about 200 mg/kg of the weight of the subject. In some embodiments, the composition comprising PIF peptide or a PIF analog or pharmaceutically acceptable salt thereof is administered in a daily dosage of up to about 150 mg/kg of the weight of the subject. In some embodiments, the composition comprising a PIF peptide or a PIF analog or pharmaceutically acceptable salt thereof is administered in a daily dosage of up to about 100 mg/kg of the weight of the subject. In some embodiments, the composition comprising a PIF peptide or a PIF analog or pharmaceutically acceptable salt thereof is administered in a daily dosage of up to about 50 mg/kg of the weight of the subject. In some embodiments, the composition comprising PIF peptide or a PIF analog or pharmaceutically acceptable salt thereof is administered in a daily dosage of up to about 25 mg/kg of the weight of the subject.

In some embodiments, the composition comprising a PIF peptide or a PIF analog or pharmaceutically acceptable salt thereof is administered in a daily dosage of up to about 10 mg/kg of the weight of the subject. In some embodiments, the composition comprising PIF peptide or a PIF analog or pharmaceutically acceptable salt thereof is administered in a daily dosage of up to about 5 mg/kg of the weight of the subject. In some embodiments, the composition comprising PIF peptide or a PIF analog or pharmaceutically acceptable salt thereof is administered in a daily dosage of up to about 1 mg/kg of the weight of the subject. In some embodiments, the composition comprising a PIF peptide or a PIF analog or pharmaceutically acceptable salt thereof is administered in a daily dosage of up to about 0.1 mg/kg of the weight of the subject. In some embodiments, the composition comprising a PIF analog or pharmaceutically acceptable salt thereof is administered in a daily dosage of up to about 0.01 mg/kg of the weight of the subject. In some embodiments, the composition comprising a PIF analog or pharmaceutically acceptable salt thereof is administered in a daily dosage of up to about 0.001 mg/kg of the weight of the subject. The dose administered to the subject can also be measured in terms of total amount of a PIF peptide or PIF analog administered per day.

In some embodiments, a subject in need thereof is administered from about 1 ng to about 500 ug of analog or pharmaceutically salt thereof per day. In some embodiments, a subject in need thereof is administered from about 1 ng to about 10 ng of analog or pharmaceutically salt thereof per day. In some embodiments, a subject in need thereof is administered from about 10 ng to about 20 ng of analog or pharmaceutically salt thereof per day. In some embodiments, a subject in need thereof is administered from about 10 ng to about 100 ng of analog or pharmaceutically salt thereof per day. In some embodiments, a subject in need thereof is administered from about 100 ng to about 200 ng of analog or pharmaceutically salt thereof per day. In some embodiments, a subject in need thereof is administered from about 200 ng to about 300 ng of analog or pharmaceutically salt thereof per day. In some embodiments, a subject in need thereof is administered from about 300 ng to about 400 ng of analog or pharmaceutically salt thereof per day. In some embodiments, a subject in need thereof is administered from about 400 ng to about 500 ng of analog or pharmaceutically salt thereof per day. In some embodiments, a subject in need thereof is administered from about 500 ng to about 600 ng of analog or pharmaceutically salt thereof per day. In some embodiments, a subject in need thereof is administered from about 600 ng to about 700 ng of analog or pharmaceutically salt thereof per day. In some embodiments, a subject in need thereof is administered from about 800 ng to about 900 ng of analog or pharmaceutically salt thereof per day. In some embodiments, a subject in need thereof is administered from about 900 ng to about 1 μg of analog or pharmaceutically salt thereof per day. In some embodiments, a subject in need thereof is administered from about 1 μg to about 100 μg of analog or pharmaceutically salt thereof per day. In some embodiments, a subject in need thereof is administered from about 100 μg to about 200 μg of analog or pharmaceutically salt thereof per day. In some embodiments, a subject in need thereof is administered from about 200 μg to about 300 μg of analog or pharmaceutically salt thereof per day. In some embodiments, a subject in need thereof is administered from about 300 μg to about 400 μg of analog or pharmaceutically salt thereof per day. In some embodiments, a subject in need thereof is administered from about 400 μg to about 500 μg of analog or pharmaceutically salt thereof per day. In some embodiments, a subject in need thereof is administered from about 500 μg to about 600 μg of analog or pharmaceutically salt thereof per day. In some embodiments, a subject in need thereof is administered from about 600 μg to about 700 μg of analog or pharmaceutically salt thereof per day. In some embodiments, a subject in need thereof is administered from about 800 μg to about 900 μg of analog or pharmaceutically salt thereof per day. In some embodiments, a subject in need thereof is administered from about 900 μg to about 1 mg of analog or pharmaceutically salt thereof per day.

In some embodiments, a subject in need thereof is administered from about 0.0001 to about 3000 milligrams of a PIF peptide or PIF analog or pharmaceutically salt thereof per day. In some embodiments, a subject is administered up to about 2000 milligrams of a PIF peptide or PIF analog or pharmaceutically salt thereof day. In some embodiments, a subject is administered up to about 1800 milligrams of a PIF peptide or PIF analog or pharmaceutically salt thereof per day. In some embodiments, a subject is administered up to about 1600 milligrams of a PIF peptide or PIF analog or pharmaceutically salt thereof per day. In some embodiments, a subject is administered up to about 1400 milligrams of a PIF peptide or PIF analog or pharmaceutically salt thereof per day. In some embodiments, a subject is administered up to about 1200 milligrams of a PIF peptide or PIF analog or pharmaceutically salt thereof per day. In some embodiments, a subject is administered up to about 1000 milligrams of a PIF peptide or PIF analog or pharmaceutically salt thereof per day. In some embodiments, a subject is administered up to about 800 milligrams of a PIF peptide or PIF analog or pharmaceutically salt thereof per day. In some embodiments, a subject is administered from about 0.0001 milligrams to about 700 milligrams of a PIF peptide or PIF analog or pharmaceutically salt thereof per dose. In some embodiments, a subject is administered up to about 700 milligrams of a PIF peptide or PIF analog or pharmaceutically salt thereof per dose. In some embodiments, a subject is administered up to about 600 milligrams of a PIF peptide or PIF analog or pharmaceutically salt thereof per dose. In some embodiments, a subject is administered up to about 500 milligrams of a PIF peptide or PIF analog or pharmaceutically salt thereof per dose. In some embodiments, a subject is administered up to about 400 milligrams of a PIF peptide or PIF analog or pharmaceutically salt thereof per dose. In some embodiments, a subject is administered up to about 300 milligrams of a PIF peptide or PIF analog or pharmaceutically salt thereof per dose. In some embodiments, a subject is administered up to about 200 milligrams of a PIF peptide or PIF analog or pharmaceutically salt thereof per dose. In some embodiments, a subject is administered up to about 100 milligrams of a PIF peptide or PIF analog or pharmaceutically salt thereof per dose. In some embodiments, a subject is administered up to about 50 milligrams of a PIF peptide or PIF analog or pharmaceutically salt thereof per dose. In some embodiments, a subject is administered up to about 25 milligrams of a PIF peptide or PIF analog or pharmaceutically salt thereof per dose. In some embodiments, a subject is administered up to about 15 milligrams of a PIF peptide or PIF analog or pharmaceutically salt thereof per dose.

In some embodiments, a subject is administered up to about 10 milligrams of a PIF peptide or PIF analog or pharmaceutically salt thereof per dose. In some embodiments, a subject is administered up to about 5 milligrams of a PIF peptide or PIF analog or pharmaceutically salt thereof per dose. In some embodiments, a subject is administered up to about 1 milligram of a PIF peptide or PIF analog or pharmaceutically salt thereof per dose. In some embodiments, a subject is administered up to about 0.1 milligrams of a PIF peptide or PIF analog or pharmaceutically salt thereof per dose. In some embodiments, a subject is administered up to about 0.001 milligrams of a PIF peptide or PIF analog or pharmaceutically salt thereof per dose.

The dose administered to the subject can also be measured in terms of total amount of a PIF peptide or PIF analog or pharmaceutically salt thereof administered per ounce of liquid prepared. In some embodiments, the PIF peptide or PIF analog or pharmaceutically salt thereof is at a concentration of about 2.5 grams per ounce of solution. In some embodiments, the PIF peptide or PIF analog or pharmaceutically salt thereof is at a concentration of about 2.25 grams per ounce of solution. In some embodiments, the PIF peptide or PIF analog or pharmaceutically salt thereof is at a concentration of about 2.25 grams per ounce of solution. In some embodiments, the PIF peptide or PIF analog or pharmaceutically salt thereof is at a concentration of about 2.0 grams per ounce of solution. In some embodiments, the PIF peptide or PIF analog or pharmaceutically salt thereof is at a concentration of about 1.9 grams per ounce of solution. In some embodiments, the PIF peptide or PIF analog or pharmaceutically salt thereof is at a concentration of about 1.8 grams per ounce of solution. In some embodiments, the PIF analog or pharmaceutically salt thereof is at a concentration of about 1.7 grams per ounce of solution. In some embodiments, the PIF peptide or PIF analog or pharmaceutically salt thereof is at a concentration of about 1.6 grams per ounce of solution. In some embodiments, the PIF peptide or PIF analog or pharmaceutically salt thereof is at a concentration of about 1.5 grams per ounce of solution. In some embodiments, the PIF peptide or PIF analog or pharmaceutically salt thereof is at a concentration of about 1.4 grams per ounce of solution. In some embodiments, the PIF peptide or PIF analog or pharmaceutically salt thereof is at a concentration of about 1.3 grams per ounce of solution. In some embodiments, the PIF peptide or PIF analog or pharmaceutically salt thereof is at a concentration of about 1.2 grams per ounce of solution. In some embodiments, the PIF peptide or PIF analog or pharmaceutically salt thereof is at a concentration of about 1.1 grams per ounce of solution. In some embodiments, the PIF peptide or PIF analog or pharmaceutically salt thereof is at a concentration of about 1.0 grams per ounce of solution. In some embodiments, the PIF peptide or PIF analog or pharmaceutically salt thereof is at a concentration of about 0.9 grams per ounce of solution. In some embodiments, the PIF peptide or PIF analog or pharmaceutically salt thereof is at a concentration of about 0.8 grams per ounce of solution. In some embodiments, the PIF peptide or PIF analog or pharmaceutically salt thereof is at a concentration of about 0.7 grams per ounce of solution. In some embodiments, the PIF peptide or PIF analog or pharmaceutically salt thereof is at a concentration of about 0.6 grams per ounce of solution. In some embodiments, the PIF peptide or PIF analog or pharmaceutically salt thereof is at a concentration of about 0.5 grams per ounce of solution. In some embodiments, the PIF peptide or PIF analog or pharmaceutically salt thereof is at a concentration of about 0.4 grams per ounce of solution. In some embodiments, the PIF peptide or PIF analog or pharmaceutically salt thereof is at a concentration of about 0.3 grams per ounce of solution. In some embodiments, the PIF peptide or PIF analog or pharmaceutically salt thereof is at a concentration of about 0.2 grams per ounce of solution. In some embodiments, the PIF peptide or PIF analog or pharmaceutically salt thereof is at a concentration of about 0.1 grams per ounce of solution. In some embodiments, the PIF peptide or PIF analog or pharmaceutically salt thereof is at a concentration of about 0.01 grams per ounce of solution. In some embodiments, the PIF peptide or PIF analog or pharmaceutically salt thereof is at a concentration of about 0.001 grams per ounce of solution prepared. In some embodiments, the PIF peptide or PIF analog or pharmaceutically salt thereof is at a concentration of about 0.0001 grams per ounce of solution prepared. In some embodiments, the PIF peptide or PIF analog or pharmaceutically salt thereof is at a concentration of about 0.00001 grams per ounce of solution prepared. In some embodiments, the PIF peptide or PIF analog or pharmaceutically salt thereof is at a concentration of about 0.000001 grams per ounce of solution prepared.

Dosage may be measured in terms of mass amount of analog per liter of liquid formulation prepared. One skilled in the art can increase or decrease the concentration of the analog in the dose depending upon the strength of biological activity desired to treat or prevent any above-mentioned disorders associated with the treatment of subjects in need thereof. For instance, some embodiments of the invention can include up to 0.00001 grams of analog per 5 mL of liquid formulation and up to about 10 grams of analog per 5 mL of liquid formulation.

In some embodiments, the pharmaceutical compositions of the claimed invention comprise at least one or a plurality of active agents other than the PIF peptide, analog of pharmaceutically acceptable salt thereof. In some embodiments, the active agent is covalently linked to the PIF peptide or PIF analog disclosed herein optionally by a protease cleavable linker (including by not limited to Pro-Pro or Cituline-Valine di-alpha-amino acid linkers). In some embodiments, the one or plurality of active agents is one or a combination of compounds chosen from: an anti-inflammatory compound, α-adrenergic agonist, antiarrhythmic compound, analgesic compound, and an anesthetic compound.

Examples of anti-inflammatory compounds include: aspirin, celecoxib, diclofenac, diflunisal, etodolac, ibuprofen, indomethacin, ketoprofen, ketorolac, nabumetone, naproxen, oxaprozin, piroxicam, salsalate, sulindac, and tolmetin.

Examples of α-adrenergic agonists include: Methoxamine, Methylnorepinephrine, Midodrine, Oxymetazoline, Metaraminol, Phenylephrine, Clonidine (mixed α2-adrenergic and imidazoline-I1 receptor agonist), Guanfacine, (preference for α2A-subtype of adrenoceptor), Guanabenz (most selective agonist for α2-adrenergic as opposed to imidazoline-I1), Guanoxabenz (metabolite of guanabenz), Guanethidine (peripheral α2-receptor agonist), Xylazine, Tizanidine, Medetomidine, Methyldopa, Fadolmidine, and Dexmedetomidine.

Examples of antiarrhythmic compound include: Amiodarone (Cordarone, Pacerone), Bepridil Hydrochloride (Vascor), Disopyramide (Norpace), Dofetilide (Tikosyn), Dronedarone (Multaq),Flecainide (Tambocor), I butilide (Corvert), Lidocaine (Xylocaine), Procainamide (Procan, Procanbid), Propafenone (Rythmol), Propranolol (Inderal), Quinidine (many trade names), Sotalol (Betapace), and Tocainide (Tonocarid).

Examples of analgesic compound include: codeine, hydrocodone (Zohydro ER), oxycodone (OxyContin, Roxicodone), methadone, hydromorphone (Dilaudid, Exalgo), morphine (Avinza, Kadian, MSIR, MS Contin), and fentanyl (Actiq, Duragesic).

Examples of anesthetic compounds include: Desflurane, Isoflurane, Nitrous oxide, Sevoflurane, and Xenon.

The compounds of the present disclosure can also be administered in combination with other active ingredients, such as, for example, adjuvants, or other compatible drugs or compounds where such combination is seen to be desirable or advantageous in achieving the desired effects of the methods described herein.

### Methods

The methods disclosed herein can be used with any of the compounds, compositions, preparations, and kits disclosed herein.

The disclosure relates to methods for treating a motor neuron disease (MND), such as amyotrophic lateral sclerosis (ALS), comprising administering an effective amount of the compositions described herein to a subject in need thereof.

In some embodiments, the composition is administered once a day to a subject in need thereof. In another embodiment, the composition is administered every other day, every third day or once a week. In another embodiment, the composition is administered twice a day. In still another embodiment, the composition is administered three times a day or four times a day. In a further embodiment, the composition is administered at least once a day for at least 1, 2, 3, 4, 5, 6, 7, 8, 9, 10, 11 or 12 weeks. In still a further embodiment, the composition is administered at least once a day for a longer term such as at least 4, 6, 8, 10, 12 or 24 months. Administration in some embodiments includes but is not limited to a dosage of 10-50 mg of composition at a frequency of minimum 1, 2, 3 or 4 times per day. Optionally, administration continues until all symptoms are resolved and cleared by medical personnel via standardized testing such as SCAT 2.

In some embodiments, the composition is administered within 1, 2, 3, 5 or 7 days of the one or more motor neuron disease. In other embodiments, the composition is administered within 1, 2, 3, 5, or 7 days of the appearance of symptoms of a motor neuron disease.

In some embodiments, the composition is administered at least once a day until the condition has ameliorated to where further treatment is not necessary. In another embodiment, the composition is administered until all symptoms of the one or more motor neuron disease (MND) are resolved. In another embodiment, the composition is administered until the subject

In some embodiments, the composition is administered for at least 1, 2, 3, 6, 8, 10, 12, or 24 months after the subject is asymptomatic.

The compositions of the present disclosure are useful and effective when administered to treat a motor neuron disease, particularly amyotrophic lateral sclerosis (ALS).

The amount of each component present in the composition will be the amount that is therapeutically effective, i.e., an amount that will result in the effective treatment of the condition (e.g., amyotrophic lateral sclerosis) when administered. The therapeutically effective amount will vary depending on the subject and the severity and nature of the injury and can be determined routinely by one of ordinary skill in the art.

This disclosure incorporates by reference in their entireties U.S. Pat. Nos. 8,222,211, 7,723,289, 7,723,290, 8,454,967, 9,097,725, 7,273,708, 7,695,977, 7,670,852, 7,670,851, 7,678,582, 7,670,850, 8,012,700, 7,789,289, 7,723,290, 8,222,211, and 8,454,967.

In some embodiments, the disclosure relates to a method of treating motor neuron disease by administering at least one or a plurality of compositions disclosed herein comprising PIF peptide, an analog thereof, or a pharmaceutically acceptable salt thereof.

In some embodiments, the disclosure relates to a method of treating motor neuron disease by administering a therapeutically effective amount or dose of one or a plurality of compositions disclosed herein comprising at least one PIF peptide, an analog thereof, or a pharmaceutically acceptable salt thereof.

In some embodiments, the disclosure relates to a method of treating motor neuron disease by administration of a pharmaceutical composition comprising a therapeutically effective amount or dose of at least one PIF peptide, an analog thereof, or a pharmaceutically acceptable salt thereof, and a pharmaceutically acceptable carrier.

In some embodiments, the disclosure relates to a pharmaceutical composition comprising a therapeutically effective amount or dose of at least one PIF peptide, an analog thereof, or a pharmaceutically acceptable salt thereof, and a pharmaceutically acceptable carrier for the treatment of motor neuron disease.

In some embodiments, the disclosure relates to the use of a therapeutically effective amount or dose of any one or plurality of compositions disclosed herein comprising at least one PIF peptide, an analog thereof, or a pharmaceutically acceptable salt thereof, and a pharmaceutically acceptable carrier in the manufacture of a medicament for the treatment of motor neuron disease.

In some embodiments, the disclosure relates to the use of a pharmaceutical composition comprising a therapeutically effective amount or dose at least one PIF peptide, an analog thereof, or a pharmaceutically acceptable salt thereof, and a pharmaceutically acceptable carrier in the manufacture of a medicament for the treatment of motor neuron disease.

In some embodiments, the disclosure relates to a method of inducing an immunomodulation effect in a subject in need thereof, when subject has been or is suspect of having motor neuron disease.

In some embodiments, the disclosure relates to a method of treating amyotrophic lateral sclerosis by administering at least one or a plurality of compositions disclosed herein comprising PIF peptide, an analog thereof, or a pharmaceutically acceptable salt thereof.

In some embodiments, the disclosure relates to a method of treating amyotrophic lateral sclerosis by administering a therapeutically effective amount or dose of one or a plurality of compositions disclosed herein comprising at least one PIF peptide, an analog thereof, or a pharmaceutically acceptable salt thereof.

In some embodiments, the disclosure relates to a method of treating amyotrophic lateral sclerosis by administration of a pharmaceutical composition comprising a therapeutically effective amount or dose of at least one PIF peptide, an analog thereof, or a pharmaceutically acceptable salt thereof, and a pharmaceutically acceptable carrier.

In some embodiments, the disclosure relates to a pharmaceutical composition comprising a therapeutically effective amount or dose of at least one PIF peptide, an analog thereof, or a pharmaceutically acceptable salt thereof, and a pharmaceutically acceptable carrier for the treatment of amyotrophic lateral sclerosis.

In some embodiments, the disclosure relates to the use of a therapeutically effective amount or dose of any one or plurality of compositions disclosed herein comprising at least one PIF peptide, an analog thereof, or a pharmaceutically acceptable salt thereof, and a pharmaceutically acceptable carrier in the manufacture of a medicament for the treatment of amyotrophic lateral sclerosis

In some embodiments, the disclosure relates to the use of a pharmaceutical composition comprising a therapeutically effective amount or dose at least one PIF peptide, an analog thereof, or a pharmaceutically acceptable salt thereof, and a pharmaceutically acceptable carrier in the manufacture of a medicament for the treatment of amyotrophic lateral sclerosis.

In some embodiments, the disclosure relates to a method of inducing an immunomodulation effect in a subject in need thereof, when subject has been or is suspect of having amyotrophic lateral sclerosis by administering at least one or a plurality of compositions disclosed herein comprising PIF peptide, an analog thereof, or a pharmaceutically acceptable salt thereof.

### Kits

According to some embodiments of the invention, the formulation may be supplied as part of a kit. In some embodiments, the kit comprises comprising a PIF peptide and/or a PIF analog or pharmaceutically acceptable salt thereof, the PIF peptide and/or a PIF analog or pharmaceutically acceptable salt thereof comprises a non-natural amino acid or is at least 70% homologous to SEQ ID NO:20. In some embodiments, the PIF peptide is a peptide comprising an amino acid sequence as described herein, such as but not limited to SEQ ID NO: 13. In another embodiment, the kit comprises a pharmaceutically acceptable salt of an analog with a rehydration mixture. In another embodiment, the pharmaceutically acceptable salt of an analog are in one container while the rehydration mixture is in a second container. The rehydration mixture may be supplied in dry form, to which water or other liquid solvent may be added to form a suspension or solution prior to administration. Rehydration mixtures are mixtures designed to solubilize a lyophilized, insoluble salt of the invention prior to administration of the composition to a subject takes at least one dose of a purgative. In another embodiment, the kit comprises a pharmaceutically acceptable salt in orally available pill form.

The kit may contain two or more containers, packs, or dispensers together with instructions for preparation and administration. In some embodiments, the kit comprises at least one container comprising the pharmaceutical composition or compositions described herein and a second container comprising a means for delivery of the compositions such as a syringe. In some embodiments, the kit comprises a composition comprising an analog in solution or lyophilized or dried and accompanied by a rehydration mixture. In some embodiments, the analog and rehydration mixture may be in one or more additional containers.

The compositions included in the kit may be supplied in containers of any sort such that the shelf-life of the different components are preserved, and are not adsorbed or altered by the materials of the container. For example, suitable containers include simple bottles that may be fabricated from glass, organic polymers, such as polycarbonate, polystyrene, polypropylene, polyethylene, ceramic, metal or any other material typically employed to hold reagents or food; envelopes, that may consist of foil-lined interiors, such as aluminum or an alloy. Other containers include test tubes, vials, flasks, and syringes. The containers may have two compartments that are separated by a readily removable membrane that upon removal permits the components of the compositions to mix. Removable membranes may be glass, plastic, rubber, or other inert material.

Kits may also be supplied with instructional materials. Instructions may be printed on paper or other substrates, and/or may be supplied as an electronic-readable medium, such as a floppy disc, CD-ROM, DVD-ROM, zip disc, videotape, audio tape, or other readable memory storage device. Detailed instructions may not be physically associated with the kit; instead, a user may be directed to an internet web site specified by the manufacturer or distributor of the kit, or supplied as electronic mail.

In another embodiment, a packaged kit is provided that contains the pharmaceutical formulation to be administered, i.e., a pharmaceutical formulation containing PIF peptide and/or a PIF analog or pharmaceutically acceptable salt thereof, a container (e.g., a vial, a bottle, a pouch, an envelope, a can, a tube, an atomizer, an aerosol can, etc.), optionally sealed, for housing the formulation during storage and prior to use, and instructions for carrying out drug administration in a manner effective to treat any one or more of the indications disclosed herein. The instructions will typically be written instructions on a package insert, a label, and/or on other components of the kit.

Depending on the type of formulation and the intended mode of administration, the kit may also include a device for administering the formulation (e.g., a transdermal delivery device). The administration device may be a dropper, a swab, a stick, or the nozzle or outlet of an atomizer or aerosol can. The formulation may be any suitable formulation as described herein. For example, the formulation may be an oral dosage form containing a unit dosage of the active agent, or a gel or ointment contained within a tube. The kit may contain multiple formulations of different dosages of the same agent. The kit may also contain multiple formulations of different active agents.

The present kits will also typically include means for packaging the individual kit components, i.e., the pharmaceutical dosage forms, the administration device (if included), and the written instructions for use. Such packaging means may take the form of a cardboard or paper box, a plastic or foil pouch, etc.

This disclosure and embodiments illustrating the method and materials used may be further understood by reference to the following non-limiting examples. Examples are intended to create a context to present neurotrauma as an integrated multiprong disease and PIF's ability to address the disease locally and systemically addressing its cause not only consequences as they were to become apparent.

The following examples are merely illustrative and should not be construed as limiting the scope of the embodiments in any way as many variations and equivalents that are encompassed by these embodiments will become apparent to those skilled in the art upon reading the present disclosure.

### EXAMPLES

### Example 1. Murine Glial Progenitor Cells Transplantation and Synthetic PreImplantation Factor (sPIF) Reduces Inflammation and Early Motor Impairment in ALS Mice

### Results. mGRPs and sPIF Improve Motor Function but not Survival

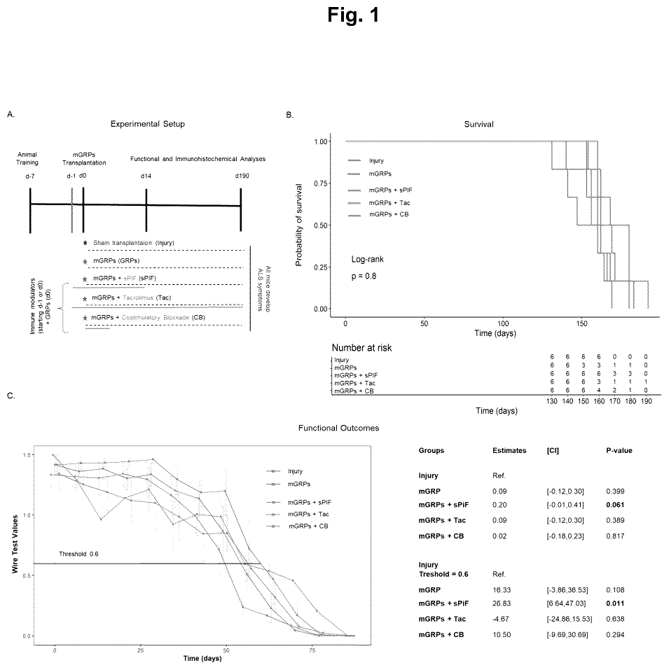

Currently, GRPs transplantation in ALS is a promising therapeutic approach and clinical trials are in preparation (ClinicalTrials.gov Identifier: NCT02478450). The present disclosure hypothesizes that mGRPs transplantation in combination with immune modulatory therapeutics will impact the graft function and therefore modulate the course of ALS. To test this hypothesis, allogenic mGRPs were transplanted into the cisterna magna using a well-defined murine ALS model of transgenic SOD1^{G93A} mice in combination with immunomodulatory drugs: sPIF, Tac, and CB (FIG. 1A). Surprisingly, the mGRPs transplantation alone did not result in increased mice survival, which is in contrast to previous reports. The addition of immunomodulatory regimes improved mice survival but did not reach statistical significance, while sPIF seemed to be superior compared to other immune modulatory drugs (FIG. 1B compare sPIF to other groups). Functional outcomes were the next focus.

Functional outcomes such as the hanging wire test are an excellent tool to detect early motor impairment, which impart quality of life. The mGRPs transplantation alone did not impact mice performance measured by hanging wire test (FIG. 1C compare mGRPs to Sham). The additional application of immune modulatory drugs resulted in a significant delay of early motor impairment in the sPIF group only (FIG. 1C compare sPIF to other groups). This difference was especially significant in the first 60 days (FIG. 1C red line at 0.6 threshold) suggesting that sPIF predominantly impacted the early phase of the injury. The grafts' survival and inflammatory responses in the brain were then investigated.

### Immunomodulatory Therapeutics Reduce Peripheral Immune Response but not Graft Survival

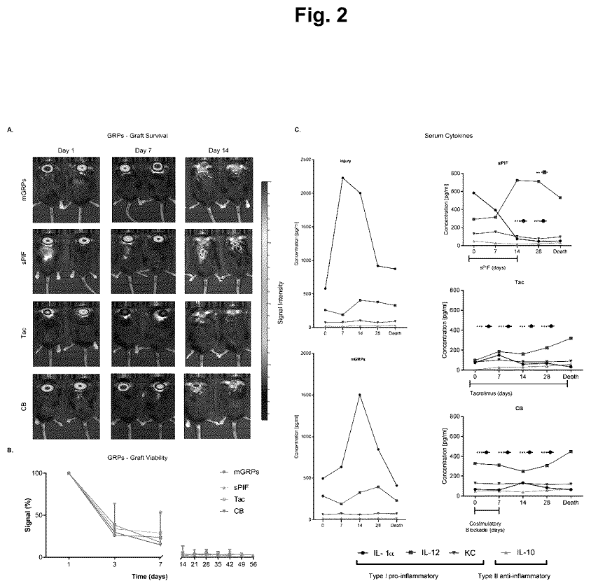

Graft survival was measured using Bioluminescent imaging first. A rapid decline of graft viability was detected during the first days with only marginal signal after day 14 (FIG. 2A and B compare mGRPs alone to other groups). This observation is in line with the lack of mGRPs effect (FIG. 1). As the application of immune modulators did not impart the grafts' survival (FIG. 2A and B), immune responses were then focused on. Notably, GRPs modulate immune responses and result in neuroprotection. As shown in FIG. 2C the SOD1^{G93A} transgenic mice showed expectantly increased peripheral pro-inflammatory IL-1α and IL-12 cytokines. The transplantation of mGRPs resulted in lower cytokines peaks but only with the additional application of immune modulators (FIG. 2C; compare sPIF, Tac and CB to Injury and mGRPs groups) decreased IL-1α peak significantly. Interestingly, in the sPIF group IL-12 plateaued at day 28 (FIG. 2C; compare sPIF to other groups), which may be due to the different immune regimes.

### mGRPs+sPIF Reduce Inflammation and Preserve Neuronal Cells

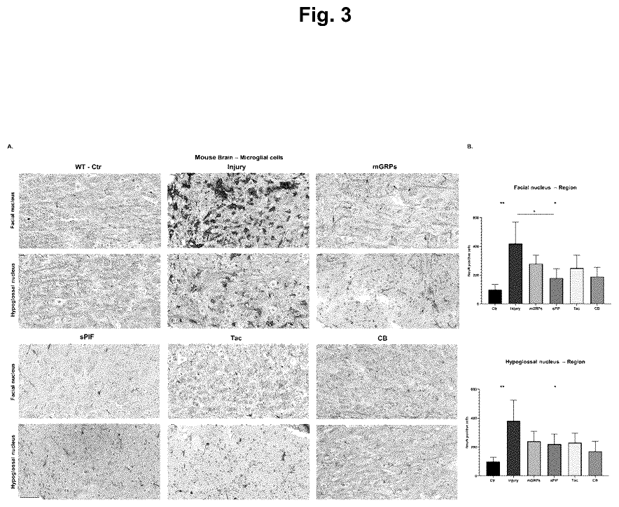

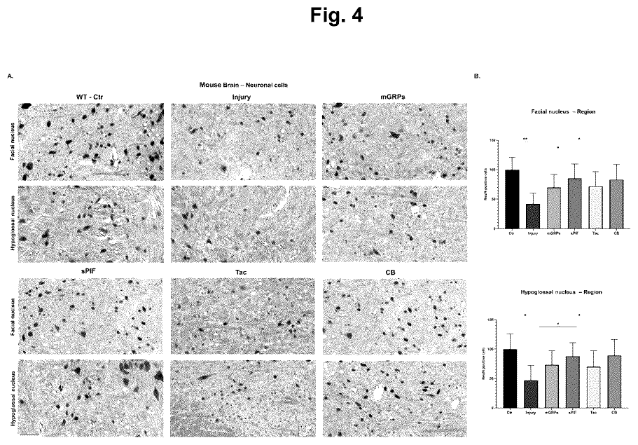

Given the functional improvements and peripheral modulation of cytokines by additional application of sPIF, Tac, and CB (FIG. 1C and 2C), the changes in the central nervous system were then evaluated. Ibal positive cells were used, as they are markers of microglial activation in the brain. Expectantly, an increased number of Iba1+ cell in both motor nuclei regions (FIG. 3A and B; compare Ctr to Injury) were detected. The transplantation of mGRPs reduced the number of Iba1+ cells but the addition of sPIF or CB resulted in significant prevention of neuroinflammation (FIG. 3A and B; compare Injury to sPIF or CB). Notably, these results are in line with previous sPIF's immune modulatory effect on the brain. Neuronal cells were then evaluated, and a reduced number of mature neurons in both motor nuclei regions (FIG. 4A and B compare Ctr to Injury) was detected. The transplantation of mGRPs restored the loss partially and again the application of both sPIF and CB resulted in significant protection of neuronal cells (FIG. 4A and B; compare Injury to sPIF or CB). These results are in line with both the modulation of peripheral (FIG. 2) and central (FIG. 3) immune responses, which are believed to be responsible for the functional improvements (FIG. 1C).

### mGRPs+sPIF Impart mGRPs Progeny

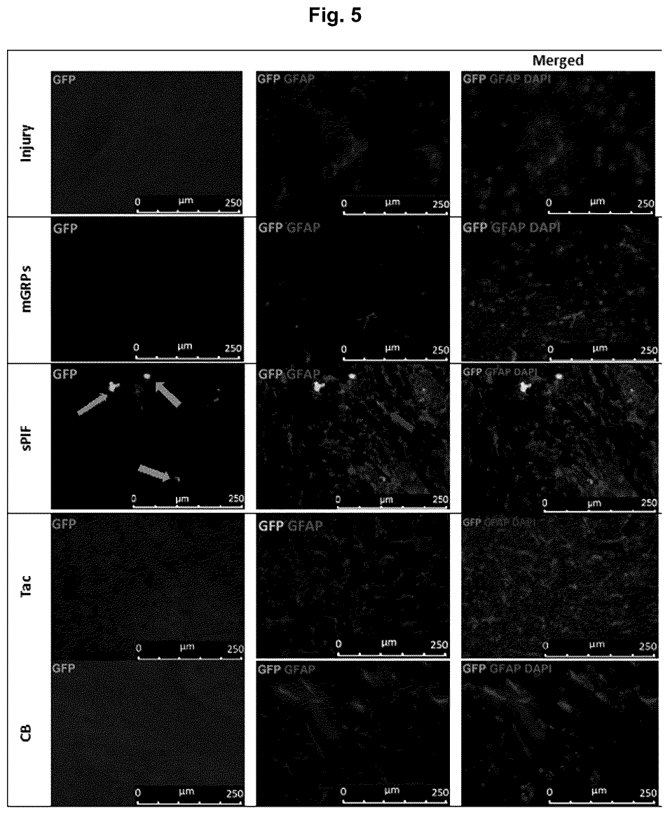

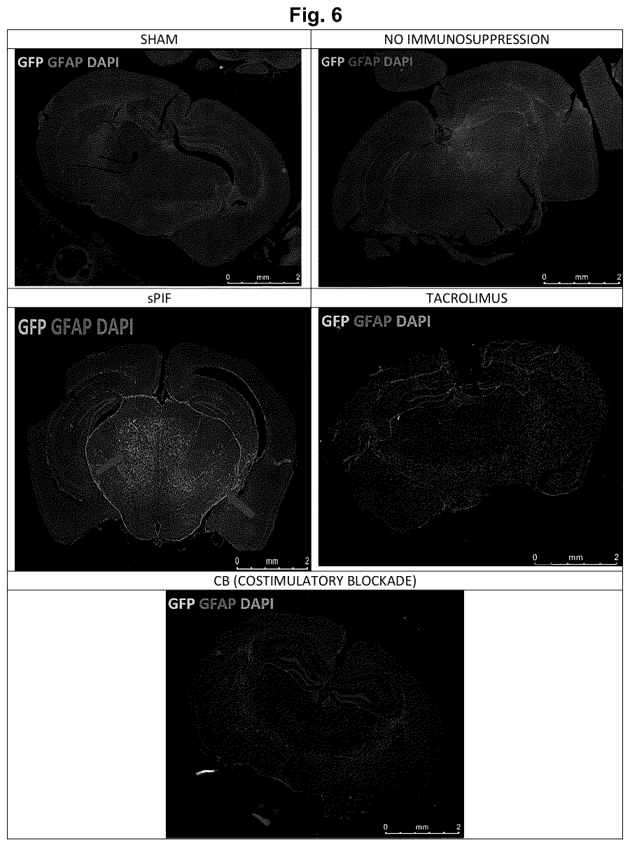

A functional improvement in mice was detected despite the rapid decline of graft viability during the first days (FIG. 1C and 2A-B compare mGRPs alone to other groups). Therefore, mGRPs progeny in the brains were detected next. Notably, glial precursors differentiate into astrocytes and oligodendrocytes and sPIF may affecting their differentiation capacity. Transplantation of mGRPs-^{GFP+} cells in the ALS transgenic mouse model (SOD1^{G93A}) resulted in few GFP+ astrocytes and oligodendrocytes in the brain parenchyma after this was evident in the sPIF group only (FIGS. 5 and 6 compare mGRPs group to other groups). This observation is in line with the detected functional improvement (FIG. 1C compare sPIF to other groups) but whether sPIF administration alone and/or in combination with mGRPs transplant is beneficial warrant further investigation.

### Discussion

The transplantation of mGRPs alone (using our therapeutic regime) didn't result in extended mice survival or functional improvement (FIG. 1). However, the first evidence that the additional use of immune modulatory drugs in ALS and especially sPIF is provided. The combination of mGRPs and sPIF modulated not only the peripheral and central immune responses (FIGS. 2 and 3) or neuronal survival (FIG. 4) but importantly improved functional outcomes as well (FIG. 1C). Given the lack of effect on graft viability (FIG. 2A and B) and progeny (FIGS. 5 and 6), the role of sPIF (alone or synergistic with mGRPs) is currently unclear. The neuroprotective effects of sPIF in both neonatal and adult murine models of brain injury open the possibility that sPIF alone may be neuroprotective in the ALS model as well. The second successful immune modulatory drug was CB. Although CB is promising, the co-stimulatory molecules are expressed prominent in mice and therefore especially mice are vulnerable to immunological co-stimulatory blockade. The use in humans is therefore not possible. Besides these modulation of innate immunity, the question of allogeneic versus semi-allogeneic transplantation and the principal role of GRPs implantation and potential histocompatibility discordance has to be considered. In such case, sPIF, as a pregnancy derived peptide, may have an advantage and further ameliorate an autoimmune syndrome.

The lack of neuroprotection after mGPRs' transplantation without immune modulatory drugs is unclear. The transplantation regime including optimal cell graft source, phenotype, dose, and method of transplantation need further investigation. The GRPs source and phenotype differ depending on the species and the therapeutic agents therefore need to be delivered and distributed broadly. The change of transplantation technique (for example use of MRI) or site (for example lumbar puncture) in combination with hydrogel-embedded vehicle may be beneficial. The increase of cell number or viability to support cell migration to distant brain areas or the co-grafting of mesenchymal stem cells or its microvesicles may further increase the efficiency. Finally, preconditioning of mGRPs prior transplantation may affect the graft function as well.

The chosen sPIF regime (starting day −1 until day 14 after mGRPs transplantation) may not be sufficient. Longer sPIF treatment would likely have a more pronounced effect on long-term functional outcomes and perhaps on mice survival. This speculation is based on the early inflammation control (until day 14) and potential correlation with decreased functional performance afterwards (FIG. 1C). The longer application as in case of Tac or preconditioning of the cell graft can be envisioned as well. This longer (or even permanent) approach may maintain the GRPs transplant and/or suppress the autoimmune anti-SOD1 protein mutant reaction. Finally, it is acknowledged that sex differences was not taken into account as the number of included animals was not sufficient and may impact the discordant results.

Altogether, the application of mGRPs imparts the course of ALS partially but the additional application of immune-modulatory therapeutics improved the neuroprotective effects significantly. The question whether sPIF alone would have been sufficient or if the mGRPs/sPIF would produce a synergistic effect needs further investigation. Given that sPIF is neuroprotective, crosses the blood-brain barrier, and already received a FAST-Track FDA approval (autoimmune diseases of non-pregnant subjects-clinicaltrials.gov, NCT02239562), the clinical use could be very promising.

### Materials and Methods

### Animals and Treatments

B6.Cg-Tg (SOD1^{G93A}) 1Gur/J (Animalab, The Jackson Laboratory, Bar Harbor, ME, USA, RRID: IMSR JAX:004435) and SCID (Animalab, Charles River Laboratory, Wilmington, MA, USA, RRID: IMSR_JAX:001303) mice were obtained from JaxMice. SOD1^{G93A} animals were divided randomly into 5 groups and SCID animals were used as control. Animals were defined (SOD1^{G93A} n=6) with sham transplantation (surgery but no transplantation) as Injury, (SOD1^{G93A} n=6) with mGRPs transplantation as (GRPs), (SOD1^{G93A} n=6) with mGRP+sPIF as (sPIF), (SOD1^{G93A} n=6) with mGRP+Tac as (Tac), and (SOD1^{G93A} n=6) with mGRP+CB (MR1+CTLA4) as (CB).

mGRP cells were received as a gift from the Department of Radiology and Radiological Science, Johns Hopkins School of Medicine (Baltimore, MD, USA). Cells were isolated as previously described from spinal cords dissected from Luc+/PLP/GFP+ mice between E12.5 and E14 stage. Briefly, cells were cultured in DMEM/F12 medium (Thermo Fisher, Waltham, MA, USA), supplemented with N2 and B27 (Life Technologies, Carlsbad, CA, USA), 1% BSA (Abcam, Cambridge, UK), Penicilin-streptomycin (Life Technologies, Carlsbad, CA, USA) and 20 ng per mL bFGF (Peprotech, Rocky Hill, NJ, USA). Cell culture flasks were covered with poly-L-lysine and laminin (Sigma, Saint Louis, MO, USA). The cells were cultured until monolayer was achieved, detached from culture flasks and characterized as previously described in terms of neural and glial cell markers, pro-neurogenic phenotype and their potential to differentiate into astrocytes. mGRP cells were detached from the culture flask with TrypLE (Thermo Fisher, Waltham, MA, USA) enzymatic digestion. Reaction was disrupted by adding PBS in 1:3 proportion. Cells were centrifuged 5 min with 1000 RPM. The viability of the mGRPs was assessed with trypan blue exclusion and only suspensions with over 70% of cell viability were used for transplantation. The suspension was prepared of 0.5 mln mGRP cells in 10 μl of PBS. Prior to surgery, acclimatization of all animals to the laboratory environment was assured and they were provided with food and water ad libitum. Controlled conditions of light (12 h light/12 h dark) and temperature (23-25° C.) for animal housing was used. Aseptic rodent survival surgery guidelines were followed. Prior to cell transplantation mice were anesthetized with 5% (induction) and maintained in 2% isoflurane. Animals were immobilized in stereotaxic device (Leica, Wetzlar, Germany). 10 μl of cell suspension was administered into cisterna magna 2 μl/min via infusion pump (KD Scientific, Hollston, MA, USA).

Synthetic PIF₁₅ (MVRIKPGSANKPSDD) was prepared by solid-phase peptide synthesis (Peptide Synthesizer, Applied Biosystems) employing Fmoc (9-fluorenylmethoxycarbonyl) chemistry at Bio-Synthesis, Inc. (Louisville, TX, USA) as previously published. Briefly, final purification was carried out by reversed-phase HPLC and the identity was verified by matrix-assisted laser desorption/ionization time-of-flight mass spectrometry (amino acid analysis at >95% purity). sPIF was applied in amount of 1 mg/kg body weight starting at day −1 prior to graft administration, and administered for 2 weeks after mGRPs transplantation (FIG. 1A).

Tacrolimus (Sigma Aldrich, St.Louis, MO, USA) was applied in amount of 1 mg/kg body weight starting at day −1 prior to grafting and administered every day until the end of observation (day 56; FIG. 1A). Costimulatory Blockade (CB) included MR1 (CD40L, Hozel Diagnostika, Bio X Cell, Lebanon, NH, USA, RRID: AB_1107601) and CTLA4 (Orencia, Bristol-Myers Squibb Company, New York, NY, USA), which were administered at amount 25 mg/kg at days 0, 2, 4 and 6 (day 0 defined as day of transplantation; FIG. 1A).

The immune modulatory regimes were chosen based on previous reports of successful immune modulation and neuroprotective property.

### Serum Collection and Cytokine Analysis

Samples were collected every week, starting from day of graft transplantation (day 0), 100 μl of blood from mouse tail. Blood was stored 1 h in room temperature, and overnight in +4° C. Serum was gently aspirated, and centrifuged 10 min with 2000 RPM to separate blood morphotic bodies. Purified serum was stored in −80° C. until further analysis. Multiplex ELISA 23-cytokine kit (M60009RDPD) was obtained from Bio-Rad (Bio-Plex Pro Mouse Cytokine 23-plex Assay #M60009RDPD, Hercules, Bio-Rad, CA, USA, RRID: AB_2857368) and performed according to instructions. 50 μl of diluted in sample diluent, 1:4 serum samples and diluted at range 1:10-1:20. Measurement standards were used, diluted 9 times according to the protocol and added at template plate. Vortexed beads were added to the assay 96-well plate. Plate was again washed 3 times with wash buffer, then from template plate, 50 μl of samples, standards and blank were transferred to measurement plate with multichannel pipette. Plate was sealed with tape and incubated 30 min. After this time, plate was washed 3 times. 25 μl of detection antibody was added to the wells with multichannel pipette. Plate was sealed and incubated for 30 min. and next washed 3 times using wash buffer. 50 μl per well Streptavidin-PE was added, with multichannel pipette. The plate was sealed, and incubated for 10 min. Wells were washed 3 times with wash buffer. Beads were resuspended with 125 μl of assay buffer and analysis was performed with Bio Plex 200 reader.

### Functional Testing and Bioluminescent Imaging (BLI) of Graft Survival

To assess the motor capacity of our treatment groups, motor skills assays were performed. Starting at day −7 SOD1^{G93A} mice (n=30) were trained in hanging wire. Animals were trained 3 times a week in 2 days interval. Weekly measurements were performed starting at day 0 until the end of the experiment. Hanging wire test was used to test neuromuscular strength and coordination. Briefly, mice were hung on a wire and had to catch the wire both by upper and lower limbs as well as tangle it with tail in order to move the whole distance towards a stick (repeated three times for each mouse and obtaining an average). Maximum points (1.5 point) were given when mice performed the task. Medium score (1 point) were given when mice were able to accomplish the whole task, but catching wire with both limbs and tail took more than 5 sec. A low score (0.66 point) was given when a mouse was able to accomplish the task only using upper limbs. A poor score (<0.66 points) was given when a mouse was unable to move the whole distance and the amount of time for which mice were able to hang on the wire (1 sec=0.016 points) was counted. All functional testing was performed in a blinded manner.

Imaging was performed at days 1, 3, 7, and then weekly. Measurements were performed using IVIS Lumina IT series III (Perkin Elmer, Waltham, Massachusetts, USA) and Xeno Light Luciferin (Perkin Elmer, Waltham, MA, USA) 150 mg per 1 kg. Optimal time enzyme activity was calculated at 10 min.

### Tissue Collection and Immunohistochemical Staining

At the end of experiment, mice were subjected to full body perfusion with 20% sucrose and 10% buffered formalin suitable for histology assays (Sigma Aldrich, St.Louis, MO, USA). Mice were decapitated and incubated in formalin for 24 h. Following the tissues (brain and spinal cord) were isolated, paraffin fixed for further analyses. After brain removal, the tissue was fixed in a formaldehyde solution (4%) for 2-4 hours at RT followed by 4° C. for a total time of 24-48 hours. Fixed tissues were embedded in paraffin and finally sectioned into 7 μm slices. After deparaffinization of the slides (xylol 3×3 min, EtOH 100% 2×3 min, EtOH 95% 1×3 min, EtOH 70% 1×3 min and dest. H₂O), the target was retrieved in Citrate Buffer (10 mM, pH=6.0) in a pressure cooker for 12 minutes and then they were let cool down for another 20 minutes. Slides were washed in 0.1% Tween-20/TBS (pH 7.6) and blocked 1 hour in 10% goat serum/1% bovine albumin/1% TritonX in TBS (pH 7.6). One part of the slides was incubated overnight with a rabbit monoclonal Neuronal antibody (NeuN, Abcam Cat #ab177487, RRID: AB_2532109) at 1:5000. Other slides were washed in 0.1% Tween-20/TBS (pH 7.6) and blocked one hour in 10% rabbit serum/1% bovine albumin/1% TritonX in TBS (pH 7.6). Iba1 antibody (Abcam Cat #ab5076, RRID: AB_2224402) at 1:100 concentration was applied on these slides during 24 hours. Following first antibody incubation, slides were washed in 0.1% Tween-20/TBS (3×5 min) and incubated in endogenous peroxidase blocking solution at RT for 15 min. Peroxidase-labeled polymer (DAKO anti rabbit or anti goat) was applied to the slides for one hour at RT. Slides were washed in in 0.1% Tween-20/TBS (3×5 min), followed by application of DAB+ chromogen in buffer substrate for 10-30 min, according to the manufacturer's instructions (DAKO EnVision+ System-HRP (DAB), K4007). Slides were rinsed in ddH₂O, counterstained in hematoxylin-eosin and dehydrated in a series of ethanol baths (95% >100%) and xylene, and mounted with Eukitt (Medite Service AG).

Assessment of neuronal and microglial positive cells were performed in the region of interest (ROI). ROI was defined as the brainstem (facial and hypoglossal) motor nuclei as these were reported to be affected in SOD1^{G93A} model previously. All images of immunohistochemical stainings were obtained with a Zeiss based microscope (Pannoramic, 3CCD camera Hitachi HV-F22CL 1,4MP and Zeiss AxioCam MRm monochrome camera) equipped with a digital camera. A 40× or 60× objective was used to acquire images for ROI evaluation. Consecutive coronal sections per animal and for each specific immunostaining were acquired by an independent observer blinded to the experimental conditions. The images were analyzed and reconstructed using Image J (US National Institutes of Health).

### Immunofluorescence Staining of the Tissues

Paraffin slides of the brain tissue after heated in 1 h in 60° C. were deparaffinized in: 1×10 min. Xylen I, 1×10 min Xylen II, 1×10 min EtOH 100%, 1×10 min EtOH 70%, 1×10 min EtOH 40%, 1×10 min dest. H₂O. The target was retrieved in Citrate Buffer+Tween 20 (10 mM, pH=6.0) in a water bath for 20 minutes in 97° C. and then they were let cool down for another 20 minutes. Slides were washed in PBS three times and blocked 1 hour in 10% Goat serum+1% bovine albumin serum. All slides was incubated overnight at 4° C. with a first primary mouse Anti-GFP antibody 1:500 (Abcam Cat #ab1218, RRID: AB_298911). Washed three times with PBS, then secondary antibody Goat Anti-Mouse IgG H&L (Alexa Fluor® 488) in concentration 1:700 (Abcam Cat #ab 150117, RRID: AB_2688012) was applied for 40 min in RT. Then one part of the slides was washed three times in PBS and the second primary Rabbit Anti-GFAP antibody (Abcam Cat #ab7260, RRID: AB_305808) was applied 1:1000, incubated overnight at 4° C. After that the secondary antibody Goat Anti-Rabbit IgG H&L (Alexa Fluor® 594) (Abcam Cat #ab 150080, RRID: AB_2650602) was applied for 40 min RT in concentration 1:700. The second part of the slides was incubated with Rabbit Recombinant Anti-Olig2 antibody (Abcam Cat #ab109186, RRID: AB_10861310) 1:100 incubated overnight in 4° C. Next day the secondary antibody Goat Anti-Rabbit IgG H&L (Alexa Fluor® 594) (Abcam Cat #ab150080, RRID: AB_2650602) was applied for 40 min RT in 1:700 concentration. All slides were washed three times in PBS and a cell nuclei were stained with DAPI (VECTASHIELD® Antifade Mounting Medium with DAPI, Vector Laboratories, Burlingame, CA, USA, Cat #H-1200, RRID: AB_2336790) for 30 min in RT. The slides were washed in PBS, mounted with DAKO mounting medium (Agilent Technologies, Santa Clara, CA, United States) and analyzed under Leica DM5500 fluorescent microscopy with LAS X software.

### Quantification and Statistical Analysis

All quantifications were performed including the manual cell counts in a blinded manner. The NeuN and Ibal were counted as positive cells in the ROI (see above) as previously reported. The number of cells was determined by unbiased counting of positive cells. The results were presented as the mean±standard deviation (mean±SD). A one-way analysis of variance (ANOVA) was used, followed by a post-hoc test (Bonferroni test) for IHC and ELISA Analyses. Considering the effect of therapy on physical activity across time, the temporal variation was described as the physical activity of each mouse group by calculating, at each time point and for each group the mean physical activity and the associated 95%-confidence interval. For the 5 groups, the effect of the therapy on the physical ability of the mice was investigated. Differences between groups were analyzed using a repeated measure ANOVA model with a fixed effect for the group, of the time and a random intercept for the mouse. In a second, more explorative, approach for each group the time when the physical activity was first measured below a certain threshold was determined. This “time to reduced activity” was then compared between groups using an ANOVA model with a fixed effect for group. The clinically relevant threshold was determined according to previous observations and defined as value 0.9 for the Wire Test. Finally, to assess difference in survival between groups, the Kaplan-Meier curves by group were used. Differences between survival curves were tested using a log-rank test. The statistical significance at p<0.05 was then determined.

All publications, patents and patent applications cited herein, whether supra or infra, are hereby incorporated by reference in their entirety to the same extent as if each individual publication, patent or patent application was specifically and individually indicated as incorporated by reference. It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein, will only be incorporated to the extent that no conflict arises between that incorporated material and the existing disclosure material.

It must be noted that, as used in this specification and the appended claims, the singular forms “a,” “an” and “the” include plural referents unless the content clearly dictates otherwise. Thus, for example, reference to a “colorant agent” includes two or more such agents.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

It should be noted that, when employed in the present disclosure, the terms “comprises,” “comprising,” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

While it is apparent that the illustrative embodiments of the invention herein disclosed fulfill the objectives stated above, it will be appreciated that numerous modifications and other embodiments may be devised by one of ordinary skill in the art. Accordingly, it will be understood that the appended claims are intended to cover all such modifications and embodiments, which come within the spirit and scope of the present invention.

What is claimed is:

1. A method of improving the clinical outcome in a subject suffering with, diagnosed with, or suspected of having motor neuron disease (MND) comprising administering to the subject a pharmaceutical composition comprising: (i) a therapeutically effective amount of preimplantation factor (PIF) peptide, an analog thereof, or a pharmaceutically acceptable salt thereof; and (ii) a pharmaceutically acceptable carrier.

2. The method of claim 1, wherein the motor neuron disease (MND) comprises amyotrophic lateral sclerosis (ALS).

3. The method of claim 1, wherein the method further comprises administering to the subject one or a combination of additional active agents chosen from: an anti-inflammatory compound, an alpha-adrenergic agonist, an antiarrhythmic compound, an analgesic compound, or an anesthetic compound.

4. The method of claim 1, wherein the PIF peptide, analog thereof, or a pharmaceutically acceptable salt thereof comprises an amino acid sequence comprising at least about 86% sequence identity to a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39 and SEQ ID NO: 40.

5. The method of claim 1, wherein the PIF peptide, analog thereof, or a pharmaceutically acceptable salt thereof comprises an amino acid sequence comprising at least about 86% sequence identity to a sequence selected from the group consisting of SEQ ID NO: 1, SEQ ID NO: 2, SEQ ID NO: 3, and SEQ ID NO: 4.

6. The method of claim 1, wherein the PIF peptide comprises an amino acid sequence comprising at least about 86% sequence identity to SEQ ID NO: 1

7. The method of claim 1, wherein the pharmaceutical composition is administered via parenteral injection, via subcutaneous injection, via intravenous injection, via intramuscular injection, via intraperitoneal injection, transdermally, orally, buccally, ocular routes, intravaginally, by inhalation, by depot injection, or by implant.

8. The method of claim 1, wherein the motor neuron diseases (MND) includes one of primary lateral sclerosis (PLS), progressive bulbar palsy or progressive muscular atrophy.

9. The method of claim 1, wherein the subject has signs selected from the group consisting of amyotrophia and/or muscular weakness, bulbar paralysis and fascicular contraction in muscles, and respiratory failure.

10. A method of reducing microglial activation in a subject suffering with, diagnosed with, or suspected of having motor neuron diseases (MND), the method comprising administration to the subject a pharmaceutical composition comprising: (i) a therapeutically effective amount of preimplantation factor (PIF) peptide, an analog thereof, or a pharmaceutically acceptable salt thereof; and (ii) a pharmaceutically acceptable carrier.

11. The method according to claim 10, wherein the administration comprises a dose of about 1 to about 5.5 mg/kg body weight.

12. The method according to claim 10, wherein the administration comprises for at least 1 week in a daily dose of about 1 to about 5.5 mg/kg/day.

13. The method according to claim 10, wherein the administering comprises establishing an initial 14-day administration period and thereafter, repeating the administration period of 12 days out of 24 days.

14. The method according to claim 10, wherein the administering of the effective amount of preimplantation factor (PIF) peptide one or a physiologically acceptable salt thereof treats a symptom or suppresses progress of the symptom by amyotrophic lateral sclerosis including at least one of a decrease in respiratory function, a spoken language disorder, dysphagia and a limb movement disorder.

15. The method of claim 10, further comprising administering a drug selected from the group consisting of riluzole and edaravone.

16. The method of claim 10, further comprising administering a non-drug treatment selected from the group consisting of invasive and non-invasive mechanical ventilation.

17. The method of claim 10, further comprising delaying disease onset in individuals at risk for disease development according to one or more predictive markers.


SEQUENCE LISTING (40 sequences)

SEQ ID NO: 1 - protein, 15 aa, synthetic construct
  REGION 1..15: note = synthetic peptide
MVRIKPGSANKPSDD

SEQ ID NO: 2 - protein, 15 aa, synthetic construct
  REGION 1..15: note = synthetic peptide
MVRIKYGSYNNKPSD

SEQ ID NO: 3 - protein, 13 aa, synthetic construct
  REGION 1..13: note = synthetic peptide
MVRIKPGSANKPS

SEQ ID NO: 4 - protein, 9 aa, synthetic construct
  REGION 1..9: note = synthetic peptide
MVRIKPGSA

SEQ ID NO: 5 - protein, 15 aa, synthetic construct
  REGION 1..15: note = synthetic peptide
GRVDPSNKSMPKDIA

SEQ ID NO: 6 - protein, 10 aa, synthetic construct
  REGION 1..10: note = synthetic peptide
SQAVQEHAST

SEQ ID NO: 7 - protein, 13 aa, synthetic construct
  REGION 1..13: note = synthetic peptide
SQAVQEHASTNMG

SEQ ID NO: 8 - protein, 13 aa, synthetic construct
  REGION 1..13: note = synthetic peptide
EVAQHSQASTMNG

SEQ ID NO: 9 - protein, 14 aa, synthetic construct
  REGION 1..14: note = synthetic peptide
GQASSAQMNSTGVH

SEQ ID NO: 10 - protein, 18 aa, synthetic construct
  REGION 1..18: note = synthetic peptide
SGIVIYQYMDDRYVGSDL

SEQ ID NO: 11 - protein, 11 aa, synthetic construct
  REGION 1..11: note = synthetic peptide
GMRELQRSANK

SEQ ID NO: 12 - protein, 9 aa, synthetic construct
  REGION 1..9: note = synthetic peptide
VIIIAQYMD

SEQ ID NO: 13 - protein, 15 aa, synthetic construct
  REGION 1..15: note = synthetic peptide
MVRIKPGSANKPSDD

SEQ ID NO: 14 - protein, 13 aa, synthetic construct
  REGION 1..13: note = synthetic peptide
SQAVQEHASTNMG

SEQ ID NO: 15 - protein, 18 aa, synthetic construct
  REGION 1..18: note = synthetic peptide
SGIVIYQYMDDRYVGSDL

SEQ ID NO: 16 - protein, 9 aa, synthetic construct
  REGION 1..9: note = synthetic peptide
MVRIKPGSA

SEQ ID NO: 17 - protein, 9 aa, synthetic construct
  REGION 1..9: note = synthetic peptide
VIIIAQYMD

SEQ ID NO: 18 - protein, 5 aa, synthetic construct
  REGION 1..5: note = synthetic peptide
MVRIK

SEQ ID NO: 19 - protein, 4 aa, synthetic construct
  REGION 1..4: note = synthetic peptide
PGSA

SEQ ID NO: 20 - protein, 15 aa, synthetic construct
  REGION 1..15: note = synthetic peptide
  VAR_SEQ 3: note = Xaa - Xaa is any amino acid except arginine
  VAR_SEQ 3: note = MISC_FEATURE - Xaa is any amino acid except arginine.
MVXIKPGSANKPSDD

SEQ ID NO: 21 - protein, 15 aa, synthetic construct
  REGION 1..15: note = synthetic peptide
  VAR_SEQ 1: note = MISC_FEATURE - Xaa is any amino acid except arginine.
XVRIKPGSANKPSDD

SEQ ID NO: 22 - protein, 15 aa, synthetic construct
  REGION 1..15: note = synthetic peptide
  VAR_SEQ 1: note = MISC_FEATURE - Xaa is any amino acid except arginine
  VAR_SEQ 3: note = MISC_FEATURE - Xaa is any amino acid except arginine
XVXIKPGSANKPSDD

SEQ ID NO: 23 - protein, 15 aa, synthetic construct
  REGION 1..15: note = synthetic peptide
  VAR_SEQ 6: note = MISC_FEATURE - Xaa is any amino acid except arginine
MVRIKXGSANKPSDD

SEQ ID NO: 24 - protein, 15 aa, synthetic construct
  REGION 1..15: note = synthetic peptide
  VAR_SEQ 4: note = MISC_FEATURE - Xaa is any amino acid except arginine
MVRXKPGSANKPSDD

SEQ ID NO: 25 - protein, 15 aa, synthetic construct
  REGION 1..15: note = synthetic peptide
  VAR_SEQ 2: note = MISC_FEATURE - Xaa is any amino acid except arginine
MXRIKPGSANKPSDD

SEQ ID NO: 26 - protein, 15 aa, synthetic construct
  REGION 1..15: note = synthetic peptide
MVRIKEGSANKPSDD

SEQ ID NO: 27 - protein, 15 aa, synthetic construct
  REGION 1..15: note = synthetic peptide
MVRGKPGSANKPSDD

SEQ ID NO: 28 - protein, 15 aa, synthetic construct
  REGION 1..15: note = synthetic peptide
MERIKPGSANKPSDD

SEQ ID NO: 29 - protein, 15 aa, synthetic construct
  REGION 1..15: note = synthetic peptide
AVRIKPGSANKPSDD

SEQ ID NO: 30 - protein, 14 aa, synthetic construct
  REGION 1..14: note = PIF mutant
  VAR_SEQ 1: note = MISC_FEATURE - Xaa = any natural or non-natural amino acid
  VAR_SEQ 3: note = MISC_FEATURE - Xaa = any natural or non-natural amino acid
XVXIKPGSANKPSD

SEQ ID NO: 31 - protein, 13 aa, synthetic construct
  REGION 1..13: note = PIF mutant
  VAR_SEQ 1: note = MISC_FEATURE - Xaa = any natural or non-natural amino acid
  VAR_SEQ 3: note = MISC_FEATURE - Xaa = any natural or non-natural amino acid
XVXIKPGSANKPS

SEQ ID NO: 32 - protein, 12 aa, synthetic construct
  REGION 1..12: note = PIF mutant
  VAR_SEQ 1: note = MISC_FEATURE - Xaa = any natural or non-natural amino acid
  VAR_SEQ 3: note = MISC_FEATURE - Xaa = any natural or non-natural amino acid
XVXIKPGSANKP

SEQ ID NO: 33 - protein, 10 aa, synthetic construct
  REGION 1..10: note = PIF mutant
  VAR_SEQ 1: note = MISC_FEATURE - Xaa = any natural or non-natural amino acid
  VAR_SEQ 3: note = MISC_FEATURE - Xaa = any natural or non-natural amino acid
XVXIKPGSAN

SEQ ID NO: 34 - protein, 10 aa, synthetic construct
  REGION 1..10: note = PIF mutant
  VAR_SEQ 1: note = MISC_FEATURE - Xaa = any natural or non-natural amino acid
  VAR_SEQ 3: note = MISC_FEATURE - Xaa = any natural or non-natural amino acid
XVXIKPGSAN

SEQ ID NO: 35 - protein, 9 aa, synthetic construct
  REGION 1..9: note = PIF mutant
  VAR_SEQ 1: note = MISC_FEATURE - Xaa = any natural or non-natural amino acid
  VAR_SEQ 3: note = MISC_FEATURE - Xaa = any natural or non-natural amino acid
XVXIKPGSA

SEQ ID NO: 36 - protein, 8 aa, synthetic construct
  REGION 1..8: note = PIF mutant
  VAR_SEQ 1: note = MISC_FEATURE - Xaa = any natural or non-natural amino acid
  VAR_SEQ 3: note = MISC_FEATURE - Xaa = any natural or non-natural amino acid
XVXIKPGS

SEQ ID NO: 37 - protein, 7 aa, synthetic construct
  REGION 1..7: note = PIF mutant
  VAR_SEQ 1: note = MISC_FEATURE - Xaa = any natural or non-natural amino acid
  VAR_SEQ 3: note = MISC_FEATURE - Xaa = any natural or non-natural amino acid
XVXIKPG

SEQ ID NO: 38 - protein, 6 aa, synthetic construct
  REGION 1..6: note = PIF mutant
  VAR_SEQ 1: note = MISC_FEATURE - Xaa = any natural or non-natural amino acid
  VAR_SEQ 3: note = MISC_FEATURE - Xaa = any natural or non-natural amino acid
XVXIKP

SEQ ID NO: 39-40 - skipped (2 entries)